(19) United States
(12) Patent Application Publication
(10) Pub. No.: US 2013/0103098 A1
(43) Pub. Date: Apr. 25, 2013

(54) Polyaxial bone anchor with pop-on shank, friction fit retainer and lateral alignment feature

(71) Applicants: Roger P. Jackson, Prairie Village, KS (US); James L. Surber, Kansas City, KS (US)

(72) Inventors: Roger P. Jackson, Prairie Village, KS (US); James L. Surber, Kansas City, KS (US)

(21) Appl. No.: 13/694,332

(22) Filed: Nov. 20, 2012

Related U.S. Application Data
(63) Continuation-in-part of application No. 13/694,110, filed on Oct. 26, 2012.
Application No. 13/694,110 is a continuation-in-part of application No. 13/573,874, filed on Oct. 10, 2012.
Application No. 13/573,874 is a continuation-in-part of application No. 13/573,516, filed on Sep. 19, 2012.
Application No. 13/573,516 is a continuation-in-part of application No. 13/573,303, filed on Sep. 7, 2012.
Application No. 13/573,303 is a continuation-in-part of application No. 13/506,365, filed on Apr. 13, 2012.
Application No. 13/506,365 is a continuation-in-part of application No. 13/385,212, filed on Feb. 8, 2012.
Application No. 13/385,212 is a continuation-in-part of application No. 13/374,439, filed on Dec. 29, 2011.
Application No. 13/374,439 is a continuation-in-part of application No. 13/373,289, filed on Nov. 9, 2011.
Application No. 13/373,289 is a continuation-in-part of application No. 13/136,331, filed on Jul. 28, 2011.
Application No. 13/136,331 is a continuation-in-part of application No. 12/924,802, filed on Oct. 5, 2010.
Application No. 12/924,802 is a continuation-in-part of application No. 12/802,849, filed on Jun. 15, 2010.
(60) Provisional application No. 61/629,617, filed on Nov. 22, 2011.
(60) Provisional application No. 61/628,222, filed on Oct. 26, 2011.
(60) Provisional application No. 61/627,374, filed on Oct. 11, 2011.
(60) Provisional application No. 61/626,250, filed on Sep. 23, 2011.
(60) Provisional application No. 61/573,508, filed on Sep. 7, 2011.
(60) Provisional application No. 61/517,088, filed on Apr. 13, 2011.
(60) Provisional application No. 61/463,037, filed on Feb. 11, 2011.
(60) Provisional application No. 61/456,649, filed on Nov. 10, 2010.
(60) Provisional application No. 61/460,234, filed on Dec. 29, 2010.
(60) Provisional application No. 61/400,504, filed on Jul. 29, 2010.
(60) Provisional application No. 61/403,915, filed on Sep. 23, 2010.
(60) Provisional application No. 61/278,240, filed on Oct. 5, 2009.
(60) Provisional application No. 61/336,911, filed on Jan. 28, 2010.
(60) Provisional application No. 61/343,737, filed on May 3, 2010.
(60) Provisional application No. 61/395,564, filed on May 14, 2010.
(60) Provisional application No. 61/395,752, filed on May 17, 2010.
(60) Provisional application No. 61/396,390, filed on May 26, 2010.
(60) Provisional application No. 61/398,807, filed on Jul. 1, 2010.
(60) Provisional application No. 61/400,504, filed on Jul. 29, 2010.
(60) Provisional application No. 61/402,959, filed on Sep. 8, 2010.
(60) Provisional application No. 61/403,696, filed on Sep. 20, 2010.
(60) Provisional application No. 61/403,915, filed on Sep. 23, 2010.
(60) Provisional application No. 61/268,708, filed on Jun. 15, 2009.
(60) Provisional application No. 61/270,754, filed on Jul. 13, 2009.
(60) Provisional application No. 61/336,911, filed on Jan. 28, 2010.
(60) Provisional application No. 61/395,564, filed on May 14, 2010.
(60) Provisional application No. 61/395,752, filed on May 17, 2010.
(60) Provisional application No. 61/396,390, filed on May 26, 2010.

(51) Int. Cl. A61B 17/86

(52) U.S. Cl. CPC A61B 17/8605
USPC 606/305

(57) ABSTRACT

A polyaxial bone screw assembly includes a threaded shank body having an integral upper portion receivable in a one piece receiver, the receiver having an upper channel for receiving a longitudinal connecting member and a lower cavity cooperating with a lower opening. A down-loadable compression insert and a friction fit split retainer are located in the receiver providing pop- or snap-on assembly of the shank with the receiver either prior to or after implantation of the shank into a vertebra. Laterally located, spaced crimping apertures in the receiver press receiver walls into engagement with the insert to prohibit rotation of the insert with respect to the receiver.

### CROSS-REFERENCE TO RELATED APPLICATIONS

This application claims the benefit of U.S. Provisional Patent Application Ser. No. 61/629,617 filed Nov. 22, 2011, that is incorporated by reference herein.

This application is also a continuation-in-part of U.S. patent application Ser. No. 13/694,110 filed Oct. 26, 2012 that claims the benefit of U.S. Provisional Patent Application Ser. No. 61/628,222 filed Oct. 26, 2011, both of which are incorporated by reference herein. This application is also a continuation-in-part of U.S. patent application Ser. No. 13/573,874 filed Oct. 10, 2012 that claims the benefit of U.S. Provisional Patent Application Ser. No. 61/627,374 filed Oct. 11, 2011, both of which are incorporated by reference herein. This application is also a continuation-in-part of U.S. patent application Ser. No. 13/573,516 filed Sep. 19, 2012 that claims the benefit of U.S. Provisional Patent Application Ser. No. 61/626,250 filed Sep. 23, 2011, both of which are incorporated by reference herein. This application is also a continuation-in-part of U.S. patent application Ser. No. 13/573,303 filed Sep. 7, 2012 that claims the benefit of U.S. Provisional Patent Application Ser. No. 61/573,508 filed Sep. 7, 2011, both of which are incorporated by reference herein. This application is also a continuation-in-part of U.S. patent application Ser. No. 13/506,365 filed Apr. 13, 2012 that claims the benefit of U.S. Provisional Patent Application Ser. No. 61/517,088 filed Apr. 13, 2011, both of which are incorporated by reference herein. This application is also a continuation-in-part of U.S. patent application Ser. No. 13/385,212 filed Feb. 8, 2012 that claims the benefit of U.S. Provisional Patent Application Ser. No. 61/463,037 filed Feb. 11, 2011, both of which are incorporated by reference herein. This application is also a continuation-in-part of U.S. patent application Ser. No. 13/374,439 filed Dec. 29, 2011 and is incorporated by reference herein. This application is also an continuation-in-part of U.S. patent application Ser. No. 13/373,289, filed Nov. 9, 2011 that claims the benefit of U.S. Provisional Patent Application Ser. No. 61/456,649 filed Nov. 10, 2010 and Provisional Patent Application Ser. No. 61/460,234 filed Dec. 29, 2010, all of which are incorporated by reference herein. This application is also a continuation-in-part of U.S. patent application Ser. No. 13/136,331 filed Jul. 28, 2011 that claims the benefit of U.S. Provisional Patent Application Ser. Nos. 61/400,504 filed Jul. 29, 2010, and 61/403,915 filed Sep. 23, 2010, all of which are incorporated by reference herein. This application is also a continuation-in-part of U.S. patent application Ser. No. 12/924,802 filed Oct. 5, 2010 that claims the benefit of the following U.S. Provisional Patent Application Ser. Nos.: 61/278,240, filed Oct. 5, 2009; 61/336,911, filed Jan. 28, 2010; 61/343,737 filed May 3, 2010; 61/395,564 filed May 14, 2010; 61/395,752 filed May 17, 2010; 61/396,390 filed May 26, 2010; 61/398,807 filed Jul. 1, 2010; 61/400,504 filed Jul. 28, 2010; 61/402,959 filed Sep. 8, 2010; 61/403,696 filed Sep. 20, 2010; and 61/403,915 filed Sep. 23, 2010, all of which are incorporated by reference herein. This application is also a continuation-in-part of U.S. patent application Ser. No. 12/802,849 filed Jun. 15, 2010 that claims the benefit of the following U.S. Provisional Patent Application Ser. Nos.: 61/268,708 filed Jun. 15, 2009; 61/270,754, filed Jul. 13, 2009; 61/336,911 filed Jan. 28, 2010; 61/395,564 filed May 14, 2010; 61/395,752 filed May 17, 2010; and 61/396,390 filed May 26, 2010, all of which are incorporated by reference herein.

### BACKGROUND OF THE INVENTION

The present invention is directed to polyaxial bone screws for use in bone surgery, particularly spinal surgery and particularly to such screws with compression or pressure inserts and expansion lock split retainers to snap over, capture and retain the bone screw shank head in the receiver member assembly and later fix the bone screw shank with respect to the receiver assembly.

Bone screws are utilized in many types of spinal surgery in order to secure various implants to vertebrae along the spinal column for the purpose of stabilizing and/or adjusting spinal alignment. Although both closed-ended and open-ended bone screws are known, open-ended screws are particularly well suited for connections to rods and connector arms, because such rods or arms do not need to be passed through a closed bore, but rather can be laid or urged into an open channel within a receiver or head of such a screw. Generally, the screws must be inserted into the bone as an integral unit along with the head, or as a preassembled unit in the form of a shank and pivotal receiver, such as a polyaxial bone screw assembly.

Typical open-ended bone screws include a threaded shank with a pair of parallel projecting branches or arms which form a yoke with a U-shaped slot or channel to receive a rod. Hooks and other types of connectors, as are used in spinal fixation techniques, may also include similar open ends for receiving rods or portions of other fixation and stabilization structure.

A common approach for providing vertebral column support is to implant bone screws into certain bones which then in turn support a longitudinal structure such as a rod, or are supported by such a rod. Bone screws of this type may have a fixed head or receiver relative to a shank thereof, or may be of a polyaxial screw nature. In the fixed bone screws, the rod receiver head cannot be moved relative to the shank and the rod must be favorably positioned in order for it to be placed within the receiver head. This is sometimes very difficult or impossible to do. Therefore, polyaxial bone screws are commonly preferred. Open-ended polyaxial bone screws typically allow for a loose or floppy rotation of the head or receiver about the shank until a desired rotational position of the receiver is achieved by fixing such position relative to the shank during a final stage of a medical procedure when a rod or other longitudinal connecting member is inserted into the receiver, followed by a locking screw or other closure. This floppy feature can be, in some cases, undesirable and make the procedure more difficult. Also, it is often desirable to insert the bone screw shank separate from the receiver or head due to its bulk which can get in the way of what the surgeon needs to do. Such screws that allow for this capability are sometimes referred to as modular polyaxial screws.

With specific reference to modular snap-on or pop-on polyaxial pedicle screw systems having shank receiver assemblies, the prior art has shown and taught the concept of the receiver and certain retainer parts forming an assembly wherein a contractile locking engagement between the parts is created to fix the shank head with respect to the receiver and retainer. The receiver and shank head retainer assemblies in the prior art have included a slotted contractile retainer ring and/or a lower pressure slotted insert with an expansion and contraction collet-type of structure having contractile locking engagement for the shank head due to direct contact between the retainer and/or the collet structure with the receiver resulting in contraction of the slotted retainer ring and/or the collet-type structure of the insert against the shank head. The receiver and slotted insert have generally included tapered locking engagement surfaces.

The prior art for modular polyaxial screw assemblies has also shown and taught that the contact surfaces on the outside of the slotted collet and/or retainer and the inside of the receiver, in addition to being tapered, can be conical, radiused, spherical, curvate, multi-curvate, rounded, as well as other configurations to create a contractile type of locking engagement for the shank head with respect to the receiver.

In addition, the prior art for modular polyaxial screw assemblies has shown and taught that the shank head can both enter and escape from a collet-like structure on the insert or from the retainer when the insert or retainer is in the up position and within an expansion recess or chamber of the receiver. This is the case unless the slotted insert and/or the slotted retainer are blocked or constrained from being able to be pushed or manipulated back up into the receiver bore or cavity, or unless the screw assemblies are otherwise uniquely configured to prevent this from happening.

### SUMMARY OF THE INVENTION

Embodiments according to the present invention differentiate from the prior art by not allowing the receiver to be removed from the shank head once the parts are snapped-on and connected. This is true even if the retainer can go back up into the expansion chamber. This approach or design has been found to be more secure and to provide more resistance to pull-out forces compared to the prior art for modular polyaxial screw designs. Collect-like structures extending downwardly from lower pressure inserts, when used in modular polyaxial screw designs, as shown in the prior art, have been found to be somewhat weak with respect to pull-out forces encountered during some spinal reduction procedures. The present invention is designed to solve these problems.

Embodiments of the present invention also differentiate from the prior art by providing a split retainer ring with inner friction fit surfaces that may include a radiused surface or surfaces that do not participate in the final locking engagement for the shank head with respect to the receiver. In addition, the retainer ring itself is uniquely characterized by a base portion providing expansion to receive and capture the shank head and then having expansion (not contraction) locking engagement between the shank head and the retainer ring base and between the retainer ring base and horizontal and vertical loading surfaces near a bottom opening of the receiver.

The expansion-only retainer ring base portion in embodiments according to the present invention is positioned entirely below the shank head hemisphere in the receiver and can be a stronger, more substantial structure to resist larger pull out forces on the assembly. The retainer ring base can also be better supported on a generally horizontal loading surface near the lower opening in the bottom of the receiver. This design has been found to be stronger and more secure when compared to that of the prior art which uses some type of contractile locking engagement between the parts, as described above; and, again, once assembled it cannot be disassembled.

Thus, a polyaxial bone screw assembly according to an embodiment of the invention includes a shank having an integral upper portion or integral radiused or spherical head and a body for fixation to a bone; a separate receiver defining an upper open channel, a central bore, a lower cavity and a lower opening; a lower compression insert; and a friction fit resilient expansion locking split retainer for capturing the shank head in the receiver lower cavity, the shank head being frictionally engaged with, but still movable in a non-floppy manner with respect to a portion of the friction fit retainer that engages a portion of the shank head prior to locking of the shank into a desired configuration. The shank is finally locked into a fixed position relative to the receiver by frictional engagement between the insert and a lower portion of the retainer, as described previously, due to a downward force placed on the compression insert by a closure top pressing on a rod, or other longitudinal connecting member, captured within the receiver bore and channel.

In an illustrated embodiment, laterally located crimping apertures are located on the receiver that cooperate with the insert to prohibit rotation of the insert with respect to the receiver during subsequent assembly with the shank. Typically, there are four crimping apertures, two on each receiver arm, located on either side of a central tooling aperture. However, fewer crimping apertures may be utilized. The crimping apertures are formed in outer surfaces of the receiver arms and do not extend completely there through. Each crimping aperture is partially defined by a crimp wall that is pressed against the insert at or near a front or rear surface of the insert, for example, after the insert is located in a desired position within the receiver, but before the shank is captured within the receiver by the resilient split retainer.

In the illustrated embodiments, retainers and compression inserts are downloaded into the receiver, but uploaded embodiments are also foreseen. The shank head can be positioned into the receiver lower cavity at the lower opening thereof prior to or after insertion of the shank into bone. In some embodiments, the compression insert may include a lock and release feature for independent locking of the polyaxial mechanism so the screw can be used like a fixed monoaxial screw. Also, in some embodiments, the shank (as well as other components of the assembly, including the closure top) can be cannulated for minimally invasive surgery applications.

Again, a pre-assembled receiver, compression insert and friction fit split retainer may be “pushed-on”, “snapped-on” or “popped-on” to the shank head prior to or after implantation of the shank into a vertebra. Such a “snapping on” procedure includes the steps of uploading the shank head into the receiver lower opening, the shank head pressing against the base portion of the split retainer ring and expanding the resilient lower open retainer portion out into an expansion portion or chamber of the receiver cavity followed by an elastic return of the retainer back to a nominal or near nominal shape thereof after the hemisphere of the shank head or upper portion passes through the lower ring-like portion of the retainer. The shank head enters into friction fit engagement with a portion of the retainer defined by an inner radiused surface of the retainer located above or near the shank hemisphere. The retainer radiused surface snaps onto the shank head as the retainer returns to a neutral or close to neutral orientation, providing a non-floppy connection between the retainer and the shank head. In the illustrated embodiments, when the shank is ultimately locked between the compression insert and the lower portion of the retainer, an upper portion of the radiused surface is moved away from the shank head located above the shank hemisphere and only an inner surface portion of the retainer located below the shank hemisphere fully engages the shank head.

The final fixation occurs as a result of a locking expansion-type of contact between the shank head and the lower portion of the split retainer and an expansion-type of non-tapered locking engagement between the lower portion of the retainer ring and the locking chamber in the lower portion of the receiver cavity. The retainer can expand more in the upper portion or expansion chamber of the receiver cavity to allow the shank head to pass through, but has restricted expansion to retain the shank head when the retainer lower ring portion is against the locking chamber surfaces in the lower portion of the receiver cavity and the shank head is forced down against the retainer ring during final locking. In some embodiments, when the polyaxial mechanism is locked, the pressure or compression insert is forced or wedged against a surface of the receiver resulting in an interference locking engagement, allowing for adjustment or removal of the rod or other connecting member without loss of a desired angular relationship between the shank and the receiver. This independent locking feature allows the polyaxial screw to function like a fixed monoaxial screw.

The lower pressure insert may also be configured to be independently locked by a tool or instrument, thereby allowing the pop-on polyaxial screw to be distracted, compressed and/or rotated along and around the rod to provide for improved spinal correction techniques. Such a tool engages the receiver from the sides and then engages outwardly extending winged arms of the insert to force or wedge the insert down into a locked position within the receiver. With the tool still in place and the correction maintained, the rod is then locked within the receiver channel by a closure top followed by removal of the tool. This process may involve multiple screws all being manipulated simultaneously with multiple tools to achieve the desired correction.

Objects of the invention further include providing apparatus and methods that are easy to use and especially adapted for the intended use thereof and wherein the tools are comparatively inexpensive to produce. Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an exploded perspective view of a polyaxial bone screw assembly according to an embodiment of the present invention including a shank, a receiver, an open friction fit retainer and a top drop and turn in place lower compression insert, further shown with a portion of a longitudinal connecting member in the form of a rod and a closure top.

FIG. 2 is an enlarged top plan view of the shank of FIG. 1.

FIG. 3 is a reduced cross-sectional view taken along the line 3-3 of FIG. 2.

FIG. 4 is an enlarged front elevational view of the receiver of FIG. 1.

FIG. 5 is a perspective view of the receiver of FIG. 4.

FIG. 6 is a side elevational view of the receiver of FIG. 4.

FIG. 7 is a bottom plan view of the receiver of FIG. 4.

FIG. 8 is a top plan view of the receiver of FIG. 4.

FIG. 9 is a cross-sectional view taken along the line 9-9 of FIG. 8.

FIG. 10 is a cross-sectional view taken along the line 10-10 of FIG. 8.

FIG. 11 is a cross-sectional view taken along the line 11-11 of FIG. 6.

FIG. 12 is an enlarged side elevational view of the retainer of FIG. 1.

FIG. 13 is a perspective view of the retainer of FIG. 12.

FIG. 14 is a top plan view of the retainer of FIG. 12.

FIG. 15 is a bottom plan view of the retainer of FIG. 12.

FIG. 16 is an enlarged bottom perspective view of the retainer of FIG. 12.

FIG. 17 is a cross-sectional view taken along the line 17-17 of FIG. 14.

FIG. 18 is a cross-sectional view taken along the line 18-18 of FIG. 14.

FIG. 19 is an enlarged perspective view of the insert of FIG. 1.

FIG. 20 is another enlarged perspective view of the insert of FIG. 1.

FIG. 21 is a side elevational view of the insert of FIG. 19.

FIG. 22 is a front elevational view of the insert of FIG. 19.

FIG. 23 is a top plan view of the insert of FIG. 19.

FIG. 24 is a bottom plan view of the insert of FIG. 19.

FIG. 25 is a cross-sectional view taken along the line 25-25 of FIG. 23.

FIG. 26 is a cross-sectional view taken along the line 26-26 of FIG. 23.

FIG. 27 is an enlarged front elevational view of the retainer and receiver of FIG. 1 with portions of the receiver broken away to show the detail thereof, the retainer being shown downloaded into the receiver (in phantom) to a tipped, partially inserted stage of assembly.

FIG. 28 is a reduced perspective view of the retainer and receiver with portions broken away, similar to what is shown in FIG. 27, showing the retainer in a subsequent stage of assembly and in a maximum state of compression as the retainer is downloaded into a lower cavity of the receiver.

FIG. 29 is an enlarged front elevational view of the retainer and receiver with portions broken away, similar to what is shown in FIG. 27, showing the retainer positioned lower in the receiver cavity than what is shown in FIG. 28 with upper spring tab arms thereof pressing resiliently against inner arms of the receiver.

FIG. 30 is a front elevational view of the retainer and receiver with portions broken away, similar to what is shown in FIG. 29, showing the retainer seated in the receiver cavity with the upper spring tab arms in a neutral state and partially extending into through apertures of the receiver, and further showing the insert of FIG. 1 in side elevation and being downwardly loaded into the receiver.

FIG. 32 is an enlarged perspective view of the retainer, receiver and insert of FIG. 31, showing the insert rotated to a desired position for assembly with the shank of FIG. 1.

FIG. 33 is a reduced perspective view of the receiver, retainer and insert of FIG. 32, showing receiver portions being crimped inwardly toward the insert.

FIG. 34 is an enlarged and partial perspective view of the receiver, retainer and insert of FIG. 33, in particular showing a receiver crimped portion engaging a front surface of the insert.

FIG. 35 is an enlarged front elevational view with portions broken away, similar to FIG. 32 and further showing the retainer after being moved upwardly into a shipping and shank receiving position wherein spring tabs thereof are pressed against the receiver and are located directly beneath arm surfaces of the insert.

FIG. 36 is a reduced front elevational view of the assembly of FIG. 35 and further showing the shank of FIG. 1 in a partial front elevational view and implanted into a portion of a vertebra, a hemisphere of the shank head and the vertebra portion are both shown in phantom.

FIG. 37 is an enlarged and partial front elevational view with portions broken away, similar to FIG. 35 further showing the shank of FIG. 36 in a stage of assembly with the receiver and retainer wherein the retainer lower portion is in an expanded state about the shank head hemisphere.

FIG. 38 is a partial front elevational view with portions broken away, similar to FIG. 37, the spherical shank upper portion or head shown fully captured by the retainer.

FIG. 39 is an enlarged and partial front elevational view with portions broken away, similar to FIG. 38, the shank upper portion with attached retainer being shown pulled down into an almost seated position within the lower receiver cavity, the retainer spring tabs in a substantially neutral state, extending outwardly and captured beneath a surface of the receiver.

FIG. 40 is a partial front elevational view with portions broken away, similar to FIG. 39 but, showing the shank upper portion fully seated within the receiver and the insert in a fully locked position pressing against the shank upper portion and in a locking interference fit engagement with the receiver.

FIG. 41 is a reduced and partial front elevational view with portions broken away, similar to FIG. 40 further showing partial assembly with the rod and closure top of FIG. 1, also shown in an enlarged and partial front elevational view.

FIG. 42 is a partial front elevational view with portions broken away, similar to FIG. 41, the rod and closure top also in a locked position within the receiver.

FIG. 43 is a reduced and partial front elevational view of the assembly of FIG. 42, but with the rod and closure top removed, the locking insert keeping the shank locked in place, and further showing an alternative deformable rod and cooperating closure top in exploded front elevational view.

FIG. 44 is an enlarged and partial front elevational view with portions broken away of the alternative assembly shown in FIG. 43, showing the deformable rod and closure top fixed within the receiver.

FIG. 45 is an enlarged and partial front elevational view with portions broken away of the assembly of FIG. 40, but showing the insert having been pulled upwardly into an unlocked position, the still fully seated retainer and shank being in non-floppy, pivotable relationship.

FIG. 46 is a reduced side elevational view of the assembly of FIG. 1, shown fully assembled with the shank disposed at a twenty-five degree (caudad) angle with respect to the receiver.

FIG. 47 is a perspective view of the assembly of FIG. 46.

FIG. 48 is an enlarged and partial side elevational view of the assembly of FIG. 46 with portions broken away to show the detail thereof.

FIG. 49 is an enlarged and partial front elevational view of the assembly of FIG. 1 with portions broken away to show the detail thereof, shown fully assembled with the shank disposed at a twenty-five degree (medial) angle with respect to the receiver.

FIG. 50 is a reduced perspective view of the assembly of FIG. 49.

FIG. 51 is an enlarged and partial front elevational view of the assembly of FIG. 1 with portions broken away to show the detail thereof, shown fully assembled with the shank disposed at a forty degree (medial) angle with respect to the receiver.

FIG. 52 is a reduced perspective view of the assembly of FIG. 51.

FIG. 53 is an enlarged perspective view of the assembly of FIG. 1 shown fully assembled and with the shank disposed at a twenty-five degree (multi-plane) angle with respect to the receiver.

FIG. 54 is an enlarged perspective view of an alternative non-locking insert for use in lieu of the locking insert shown in FIG. 1.

FIG. 55 is an enlarged and partial front elevational view (with portions broken away) of the receiver, retainer, rod and closure top of FIG. 1 shown fully assembled with the alternative insert of FIG. 54, also shown in front elevation, with portions broken away to show the detail thereof.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. It is also noted that any reference to the words top, bottom, up and down, and the like, in this application refers to the alignment shown in the various drawings, as well as the normal connotations applied to such devices, and is not intended to restrict positioning of the bone attachment structures in actual use.

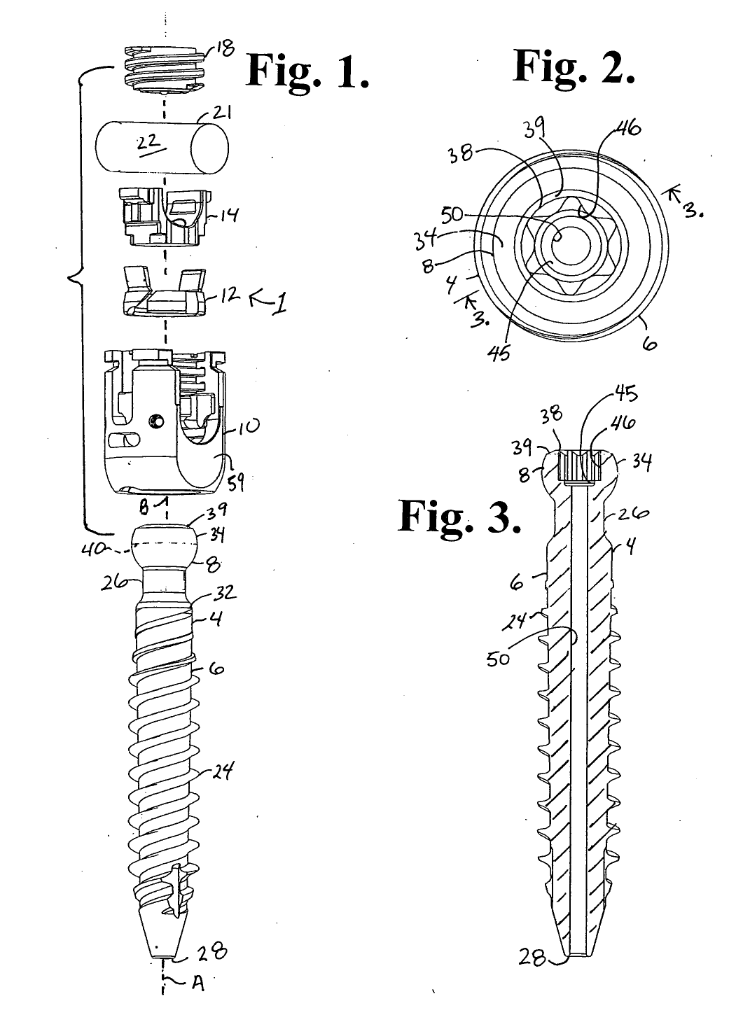

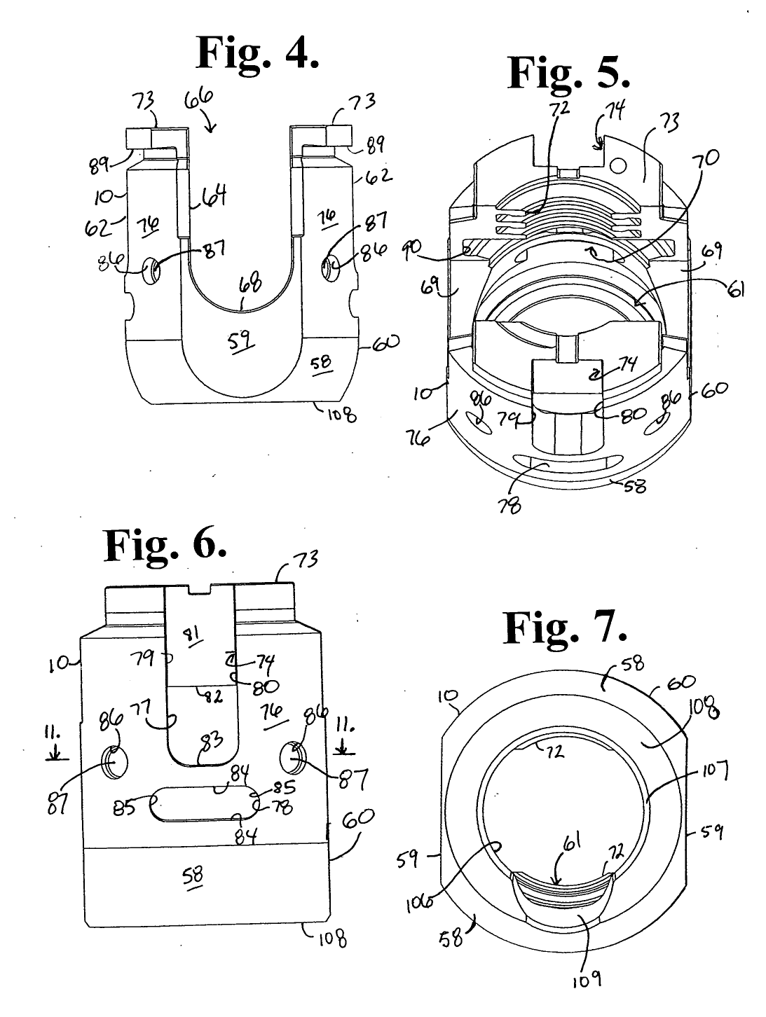

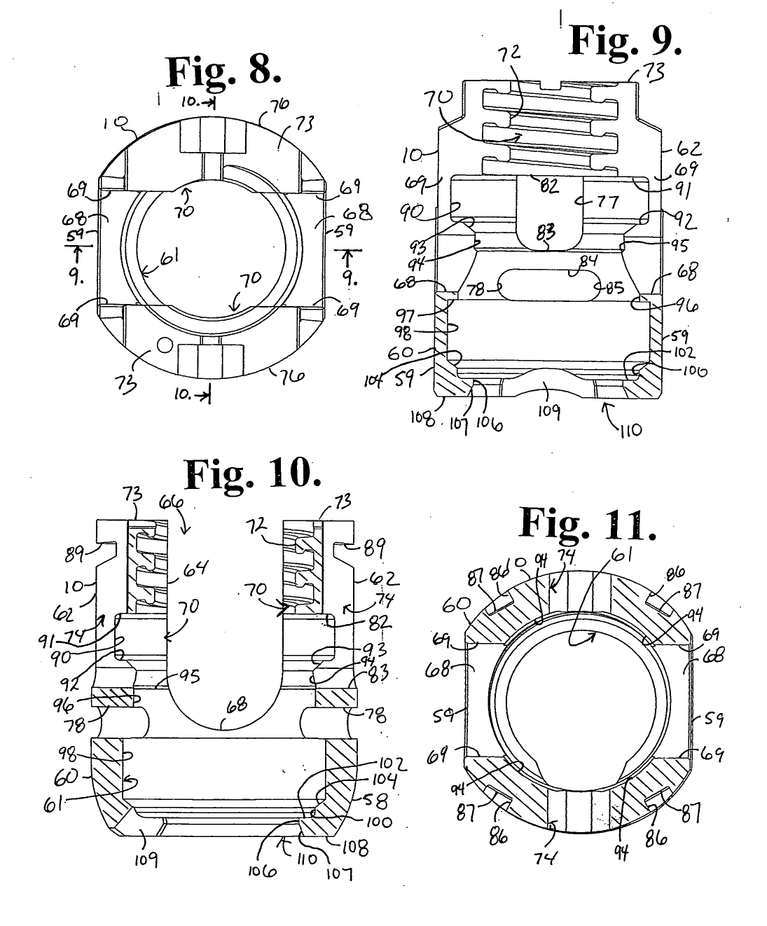

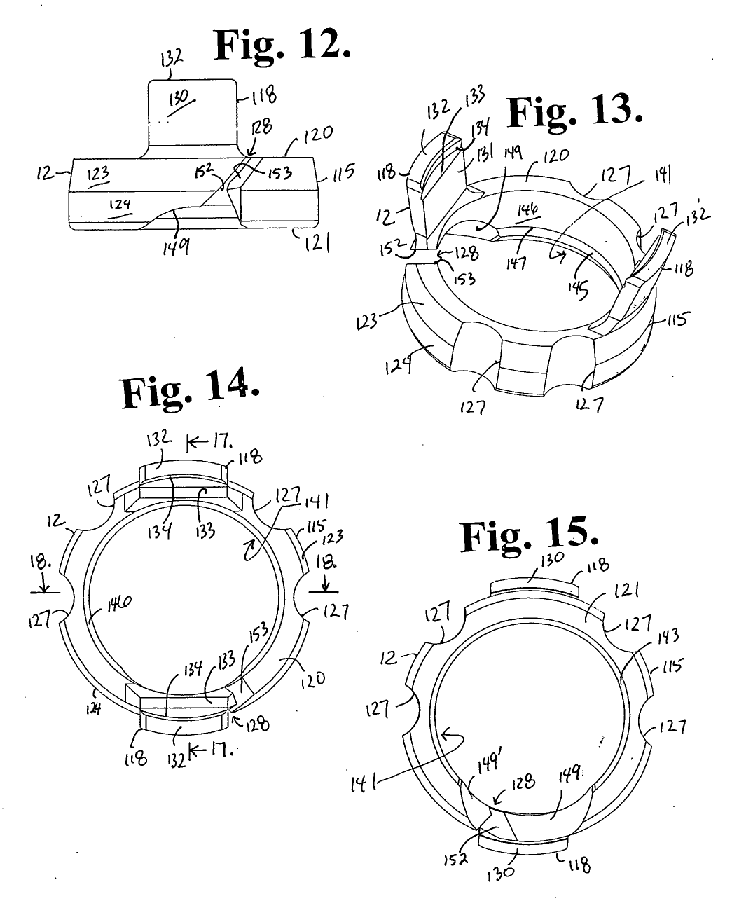

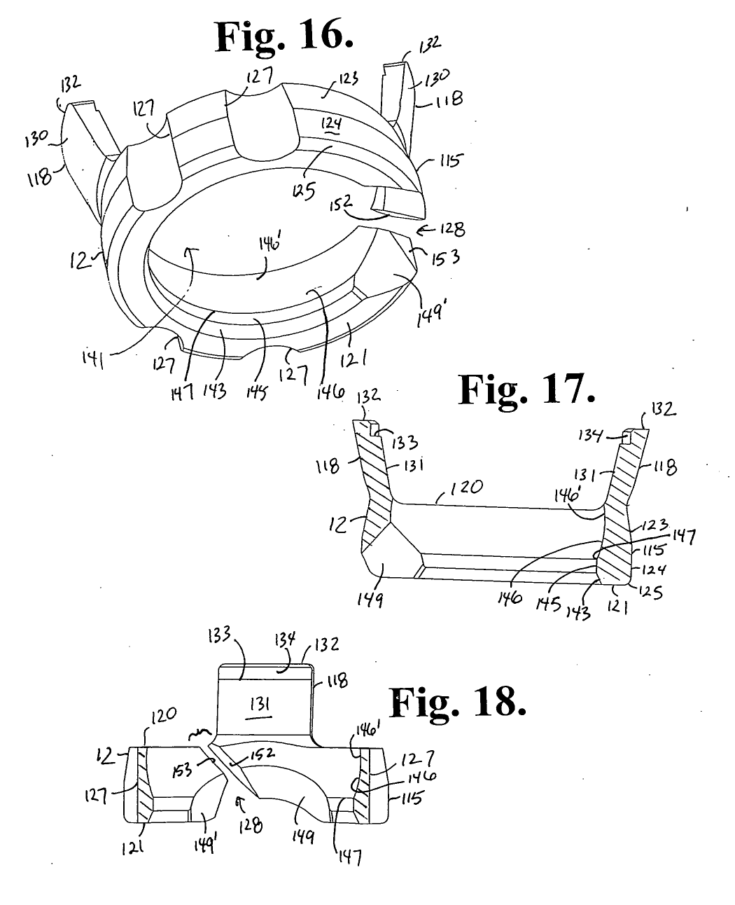

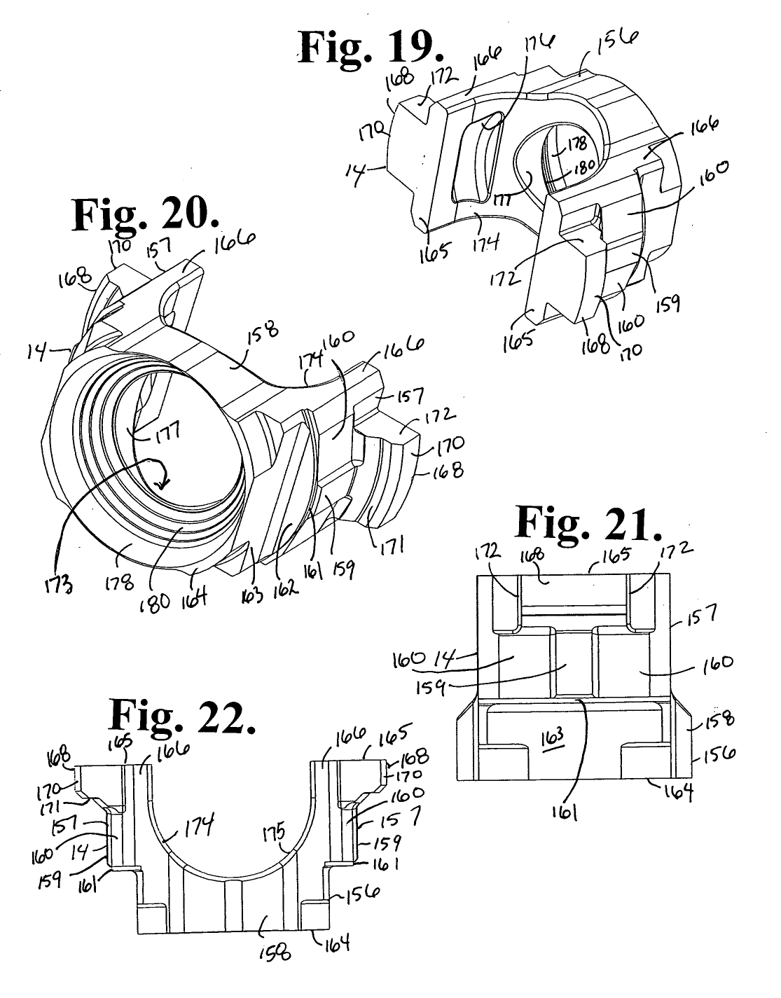

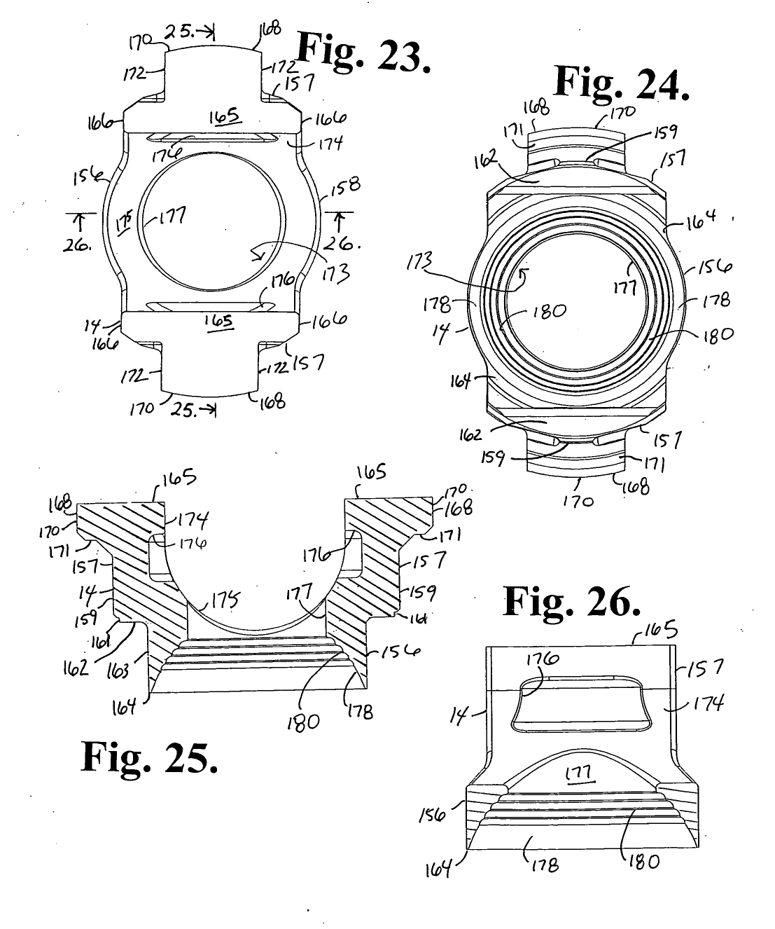

With reference to FIGS. 1-53, the reference number 1 generally represents a polyaxial bone screw apparatus or assembly according to the present invention. The assembly 1 includes a shank 4, that further includes a body 6 integral with an upwardly extending upper portion or head 8; a receiver 10; a friction fit retainer 12, and a crown-like compression or pressure insert 14. The receiver 10, retainer 12 and compression insert 14 are initially assembled and may be further assembled with the shank 4 either prior or subsequent to implantation of the shank body 6 into a vertebra 17, as will be described in greater detail below. FIGS. 1 and 41-42 further show a closure structure 18 for capturing a longitudinal connecting member, for example, a rod 21 which in turn engages the compression insert 14 that presses against the shank head 8 into fixed frictional contact with the retainer 12, so as to capture, and fix the longitudinal connecting member 21 within the receiver 10 and thus fix the member 21 relative to the vertebra 17.

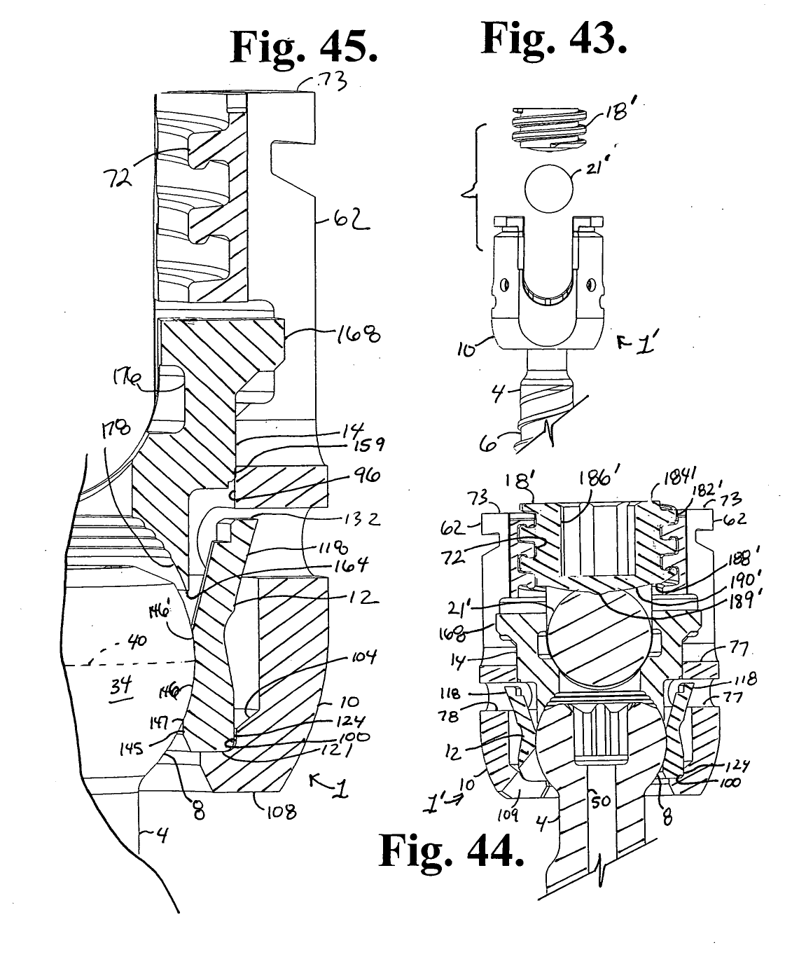

The receiver 10 and the shank 4 cooperate in such a manner that the receiver 10 and the shank 4 can be secured at any of a plurality of angles, articulations or rotational alignments relative to one another and within a selected range of angles both from side to side and from front to rear, to enable flexible or articulated engagement of the receiver 10 with the shank 4 until both are locked or fixed relative to each other near the end of an implantation procedure. The illustrated rod 21 is hard, stiff, non-elastic and cylindrical, having an outer cylindrical surface 22. In some embodiments, the rod 21 may be elastic, deformable and/or of different materials and cross-sectional geometries (see, e.g., FIGS. 43 and 44). In some embodiments of the invention (not shown), the closure top presses directly on the insert 14 when the rod is deformable.

The shank 4, best illustrated in FIGS. 1-3, is elongate, with the shank body 6 having a helically wound bone implantable thread 24 (single or dual lead thread form and different thread types) extending from near a neck 26 located adjacent to the upper portion or head 8, to a tip 28 of the body 6 and extending radially outwardly therefrom. During use, the body 6 utilizing the thread 24 for gripping and advancement is implanted into the vertebra 17 leading with the tip 28 and driven down into the vertebra with an installation or driving tool (not shown), so as to be implanted in the vertebra to a location at or near the neck 26, as shown in FIG. 31, for example, and more fully described in the paragraphs below. The shank 4 has an elongate axis of rotation generally identified by the reference letter A.

The neck 26 extends axially upward from the shank body 6. The neck 26 may be of the same or is typically of a slightly reduced radius as compared to an adjacent upper end or top 32 of the body 6 where the thread 24 terminates. Further extending axially and outwardly from the neck 26 is the shank upper portion or head 8 that provides a connective or capture apparatus disposed at a distance from the upper end 32 and thus at a distance from the vertebra 17 when the body 6 is implanted in such vertebra.

The shank upper portion 8 is configured for a pivotable connection between the shank 4 and the retainer 12 and receiver 10 prior to fixing of the shank 4 in a desired position with respect to the receiver 10. The shank upper portion 8 has an outer, convex and substantially spherical surface 34 that extends outwardly and upwardly from the neck 26 that terminates at a substantially annular, planar rim surface 38 that is perpendicular to the shank central axis A. A frusto-conical surface 39 extends from the spherical surface 34 inwardly to the top rim 38, providing additional clearance during pivoting of the shank with respect to the receiver 10 and the insert 14. The spherical surface 34 has an outer radius configured for temporary frictional, non-floppy, sliding cooperation with one or more surfaces or edges of the retainer 12, as well as ultimate frictional engagement with the retainer 12 at, at least one lower inner surface thereof and ultimate frictional engagement with the insert 14 at an inner partially spherical surface thereof and/or stepped or ridged surfaces thereof, as will be discussed more fully in the paragraphs below. In FIG. 1 and some of the other figures, a dotted line 40 designates a hemisphere of the spherical surface 34. The spherical surface 34 shown in the present embodiment is substantially smooth, but in some embodiments may include a roughening or other surface treatment and is sized and shaped for cooperation and ultimate frictional engagement with the compression insert 14 as well as ultimate frictional engagement with a lower ring-like edge of the retainer 12. The shank spherical surface 34 is locked into place exclusively by the insert 14 and the retainer 12 lower edged portion and not by inner surfaces defining the receiver cavity.

A counter sunk and stepped or graduated annular seating surface or base 45 partially defines a portion of an internal drive feature or imprint 46. In some embodiments of the invention, the surface 45 is substantially planar. The illustrated internal drive feature 46 is an aperture formed in the top 38 and has a star shape designed to receive a tool (not shown) of an Allen wrench type, into the aperture for rotating and driving the bone screw shank 4 into the vertebra 17. It is foreseen that such an internal tool engagement structure may take a variety of tool-engaging forms and may include one or more apertures of various shapes, such as a pair of spaced apart apertures or a single hex shape or other type of multi-lobular or star-shaped aperture. The graduated seat or base surfaces 45 of the drive feature 46 are disposed substantially perpendicular to the axis A with the drive feature 46 otherwise being coaxial with the axis A. As illustrated in FIGS. 2 and 3, the drive seat 45 having beveled or stepped surfaces advantageously further enhances gripping with the driving tool. In operation, the driving tool (not shown) is received in the internal drive feature 46, being seated at the base 45 and engaging the faces of the drive feature 46 for both driving and rotating the shank body 6 into the vertebra 17, either before or after the shank 4 is connected to the receiver 10 via the retainer 12, the driving tool extending into the receiver 10 when the shank 4, retainer 12 and receiver 10 combination is driven into the vertebra 17.

The shank 4 shown in the drawings is cannulated, having a small central bore 50 extending an entire length of the shank 4 along the axis A. The bore 50 is defined by an inner cylindrical wall of the shank 4 and has a circular opening at the shank tip 28 and an upper circular opening communicating with the external drive 46 at the driving seat 45. The bore 50 is coaxial with the threaded body 6 and the upper portion or head 8. The bore 50 provides a passage through the shank 4 interior for a length of wire (not shown) inserted into the vertebra 17 prior to the insertion of the shank body 6, the wire providing a guide for insertion of the shank body 6 into the vertebra 17. It is foreseen that the shank could be solid and made of different materials, including metal and non-metals.

To provide a biologically active interface with the bone, the threaded shank body 6 may be coated, perforated, made porous or otherwise treated. The treatment may include, but is not limited to a plasma spray coating or other type of coating of a metal or, for example, a calcium phosphate; or a roughening, perforation or indentation in the shank surface, such as by sputtering, sand blasting or acid etching, that allows for bony ingrowth or ongrowth. Certain metal coatings act as a scaffold for bone ingrowth. Bio-ceramic calcium phosphate coatings include, but are not limited to: alpha-tri-calcium phosphate and beta-tri-calcium phosphate (Ca₃(PO₄)₂, tetra-calcium phosphate (Ca₄P₂O₉), amorphous calcium phosphate and hydroxyapatite (Ca₁₀(PO₄)₆(OH)₂). Coating with hydroxyapatite, for example, is desirable as hydroxyapatite is chemically similar to bone with respect to mineral content and has been identified as being bioactive and thus not only supportive of bone ingrowth, but actively taking part in bone bonding.

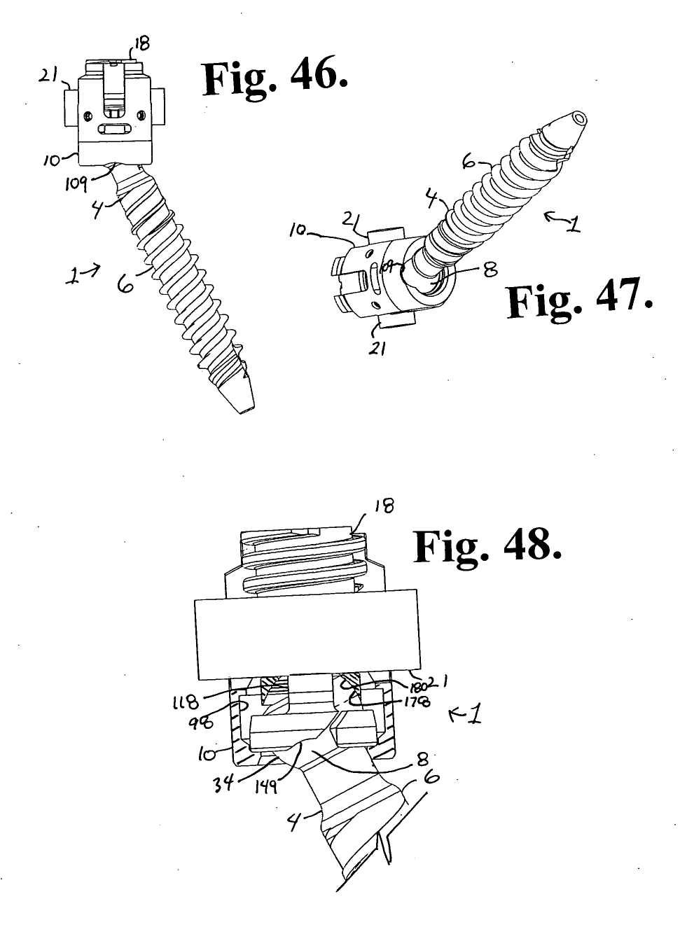

With particular reference to FIGS. 1 and 4-11, the receiver 10 has a generally U-shaped appearance with partially discontinuous cylindrical inner and outer profiles as well as planar and other curved surfaces. The receiver 10 has an axis of rotation B that is shown in FIG. 1 as being aligned with and the same as the axis of rotation A of the shank 4, such orientation being desirable, but not required during assembly of the receiver 10 with the shank 4. After the receiver 10 is pivotally attached to the shank 4, either before o r after the shank 4 is implanted in a vertebra 17, the axis B is typically disposed at an angle with respect to the axis A, as shown, for example, in FIGS. 46-53.

The receiver 10 includes a base 60 with a substantially curved or radiused lower outer surface 58 and a pair of opposed planar surface portions or flats 59. The base 60 defines a bore or inner cavity, generally 61, the base 60 being integral with a pair of opposed upstanding arms 62. The opposed flats 59 begin at the base 60 and extend upwardly to a location between the arms 62, with narrow portions of the flats 59 also extending upwardly along front and rear surfaces of each of the arms 62. The arms 62 form a cradle and define a U-shaped channel 64 between the arms 62 with an upper opening, generally 66, and a U-shaped lower channel portion or seat 68 adjacent each flat 59, the channel 64 having a width for operably snugly receiving the rod 21 or portion of another longitudinal connector, or rigid sleeve of a tensioned cord connecting member, between the arms 62, the channel 64 communicating with the base cavity 61. Inner opposed substantially planar arm surfaces 69 partially define the channel 64 above the curved seat 68 and partially define outer sides of each arm interior surface generally 70, that includes various inner cylindrical profiles, an upper one of which is a partial helically wound guide and advancement structure 72 located adjacent top surfaces 73 of each of the arms 62. In the illustrated embodiment, the guide and advancement structure 72 is a partial helically wound interlocking flangeform configured to mate under rotation with a similar structure on the closure structure 18, as described more fully below. However, it is foreseen that for certain embodiments of the invention, the guide and advancement structure 72 could alternatively be a square-shaped thread, a buttress thread, a reverse angle thread or other thread-like or non-thread-like helically wound discontinuous advancement structures, for operably guiding under rotation and advancing the closure structure 18 downward between the arms 62, as well as eventual torquing when the closure structure 18 abuts against the rod 21 or other longitudinal connecting member. It is foreseen that the arms 62 could have break-off extensions.

An opposed pair of vertically extending outer grooves, generally 74, running substantially parallel to the receiver axis B are centrally formed in outer curved (illustrated as substantially cylindrical) convex surfaces 76 of the arms 62. Each groove 74 runs centrally from the respective arm top surface 73 and terminates at a through aperture 77. Each aperture 77 extends through the respective arm surface 77 to the respective inner arm surface 70 and is located spaced from the receiver base 60. In the illustrated embodiment, each arm also includes a second, lower through aperture 78 located below and spaced from the aperture 77 and spaced from the base outer surface 58. Each groove 74 has an upper opening partially defined by a pair of opposed surfaces 79 and 80 and a substantially planar outer wall surface 81 extending between the surfaces 79 and 80. The planar wall surface terminates at the top arm surface 73 and at a lower surface 82 partially defining the aperture 77. The illustrated opposed surfaces 79 and 80 are parallel and are sized to receive portions of an elongate tool (not shown) for locking and unlocking the insert 14 to the receiver as will be described in greater detail below. In some embodiments, the surfaces 79 and 80 may be disposed at a slight angle with respect to each other, forming a dovetail-like space for maintaining a close relationship between an elongate tool (not shown) that enters into the groove 74 at the arm top surface 73 and is slidingly received between the surfaces 79 and 80. The surfaces 79 and 80 extend beyond the surface 82 and thus partially form side surfaces of the through aperture 77. The surfaces 79 and 80 terminate at a lower surface 83 that also partially defines the through aperture 77. The surface 83 is substantially perpendicular to the surfaces 79 and 80. Thus, the illustrated through aperture 77 located below each grooves 74 is substantially the same width as the groove 74 there-above, resulting in the aperture 77 having a substantially rectangular profile. The through apertures 77 are sized and shaped for receiving tooling and also the outer tangs or tabs of the retainer 12 during assembly as shown, for example, in FIG. 27.

Each of the lower through apertures 78 include substantially planar opposed top and bottom surfaces 84 and opposed C-shaped surfaces 85, the apertures 78 sized and shaped for receiving outer tabs of the retainer 12 when the retainer 12 is seated within the receiver 10 inner cavity 61 during manipulation and locking stages of the assembly 1 as will be described in greater detail below and shown, for example, in FIGS. 32 and 39-42.

With particular reference to FIGS. 6, 11 and 34, formed in the arm surfaces 76 and located on either side of the through apertures 77 and 78 are lateral crimping apertures 86. The four crimping apertures 86 are substantially circular in profile and do not extend completely through the respective arms 62, but rather terminate at a location between the arm surface 76 and the inner arm surface 70 to provide a crimping portion or wall 87. The crimping portions or walls 87 are sized and shaped for pressing or crimping some or all of the wall material inwardly onto front and rear surfaces of the insert 14 to prohibit rotation and misalignment of the insert 14 with respect to the receiver 10 as will be described in greater detail below. It is noted that although four crimping apertures are shown, in some embodiments of the invention there may be fewer or greater number of crimping apertures, for example, two crimping apertures, one at each arm 62 may be used in lieu of the illustrated four crimping apertures.

The receiver 10 is a one-piece or integral structure and is devoid of any spring tabs or collet-like structures. Preferably the insert and/or receiver are configured with structure for blocking rotation of the insert with respect to the receiver, such as the crimp walls 87, but allowing some up and down movement of the insert with respect to the receiver during the assembly and implant procedure. Also formed in each outer arm surface 76 near the top surface 73 is an undercut tool receiving and engaging groove 89. Some or all of the apertures and grooves described herein, including, but not limited to grooves 74, apertures 77 and 78, and grooves 89 may be used for holding the receiver 10 during assembly with the insert 14, the retainer 12 and the shank 4; during the implantation of the shank body 6 into a vertebra when the shank is pre-assembled with the receiver 10; during assembly of the bone anchor assembly 1 with the rod 21 and the closure structure 18; and during lock and release adjustment of inserts with respect to the receiver 10, either into or out of frictional engagement with the inner surfaces of the receiver 10 as will be described in greater detail below. It is foreseen that tool receiving grooves or apertures may be configured in a variety of shapes and sizes and be disposed at other locations on the receiver arm 62 outer surfaces 76 and/or inner surfaces 70 as well as the base 60 outer or inner surfaces.

Returning to the interior surfaces, generally 70, of the receiver arms 62, located below the discontinuous guide and advancement structure 72 is a discontinuous cylindrical surface 90 partially defining a run-out feature for the guide and advancement structure 72. Adjacent to and above the surface 90 is a discontinuous upper annular ceiling surface 91. The upper annular surface 91 includes the surface 82 that partially defines the aperture 77 and also includes bottom surfaces of the guide and advancement structure 72. Also adjacent to and below the surface 90 is a discontinuous annular surface or step 92 that in turn is adjacent to a discontinuous frusto-conical surface 93 that extends from the surface 92 inwardly toward the axis B. Adjacent the surface 93 is another substantially cylindrical discontinuous surface 94 that may in some embodiments run frusto-conical either toward or away from the axis B, depending upon, for example, clearance requirements for the top loading of the retainer 12 and/or the insert 14 and also modifying (enlarging or reducing) a thickness for the crimping walls 87, if desired. In the current embodiment, the surface 94 terminates at a small discontinuous ledge or lip 95 directed inwardly toward the axis B. The through apertures 77 extend through both the cylindrical surface 90 and the surfaces 93 and 94. A cylindrical surface 96 is adjacent to and runs downwardly from the lip 95 towards the base cavity 61. A lower portion of the cylindrical surface 96 is continuous and thus partially defines the base cavity 61. The cylindrical surface 96 has a diameter smaller than a diameter of the cylindrical surface 90, but larger than a diameter of the surface 94. The receiver inner arms 70 may further include other sloped, stepped or chamfered surfaces between the cylindrical surfaces 90, 94 and 96 as desired for ease in assembly of the other top loaded components. The lower through apertures 78 extend through the cylindrical surface 96.

The continuous portion of the inner cylindrical surface 96 terminates at a ledge surface or chamber ceiling 97 that extends outwardly from the surface 96 and away from the axis B, the surface 97 being substantially perpendicular to the axis B, but could be oblique thereto. The surface 97 is annular and defines an upper ceiling or stop of a retainer expansion portion or chamber of the inner cavity 61 that is further defined by a cylindrical surface 98 that is adjacent the surface 97. The cylindrical surface 98 has a diameter greater than the diameters of the surfaces 94 and 96. The surfaces 90, 93, 94, 96 and 98 are all centrally aligned with the receiver axis B. The surface 98 defines a circumferential recess that is sized and shaped to receive the retainer 12 lower portion as it expands around the shank upper portion 8 as the shank 8 moves upwardly toward the channel 64 during assembly. It is foreseen that the recess could be tapered or conical in configuration.

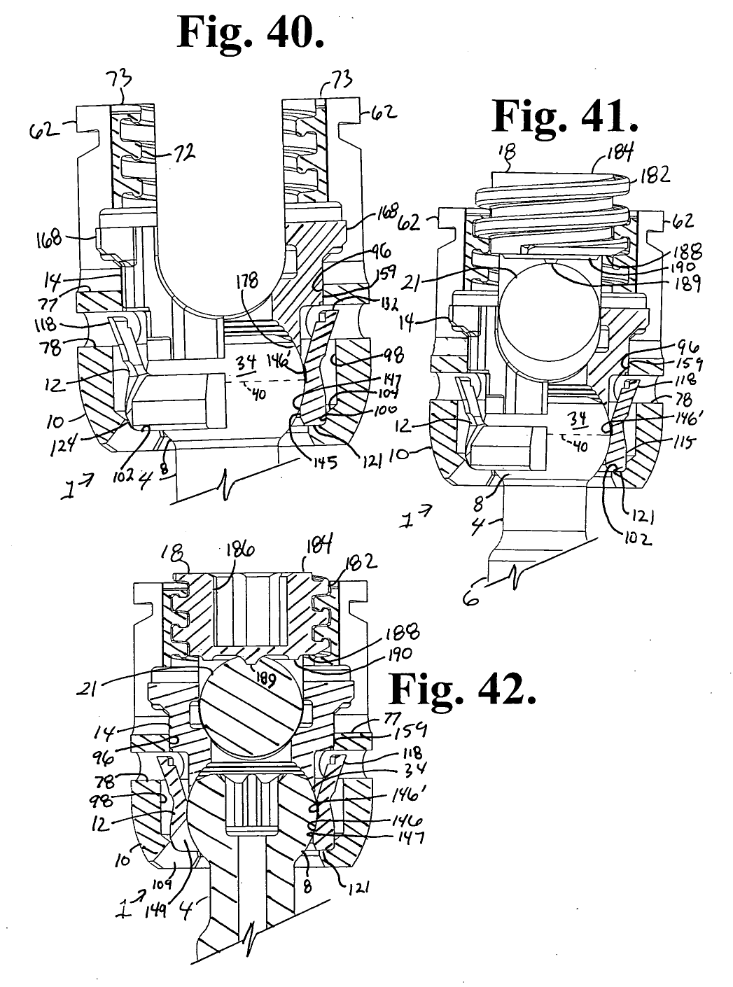

A cylindrical surface 100 adjacent to an annular lower step surface 102 provide a lower seat for the retainer 12 as will be described in greater detail below. The surface 102 is are substantially perpendicular to the surface 100 and the receiver axis B. The surfaces 100 and 102 are located below the cylindrical surface 98 in the lower part of the base 60 and are sized and shaped to closely receive and surround a lower base portion and lower skirt or sub-structure of the retainer 12 when the retainer is in a nominal or reduced deployment position as shown in FIG. 40, for example. Thus, the cylindrical surface 100 has a diameter smaller than the diameter of the cylindrical surface 98 that defines the expansion area or expansion chamber for the retainer 12. The surface 100 is joined or connected to the surface 98 by one or more beveled, curved or conical transition step surfaces 104. In the illustrated embodiment, a single frusto-conical step surface 104 is located between the surface 98 and the surface 100. The surface 104 allows for sliding and nominal or deployment positioning of the retainer 12 into the space defined by the surface 100 and ultimate seating of the retainer 12 on the lower substantially horizontal annular surface 102.

Located below and adjacent to the annular seating surface 102 is a lower cylindrical, edge or rim surface 106 that communicates with a beveled or flared bottom opening surface 107, the surface 107 communicating with an exterior base or bottom surface 108 of the base 60, defining a lower opening, generally 110, into the base cavity 61 of the receiver 10. In various embodiments of the invention, one or more curvate cut-out or cupped surfaces may be formed in a portion of the base surface 108, as well as in portions of the surfaces 107, 106 and 100-104, the cupped surface being typically located substantially centrally and directly below an arm 62. Such a cupped surface may be sized and shaped for providing clearance for an increased angle of articulation between the shank 4 and the receiver 10. In the present embodiment, one such concave or cupped surface 109 is illustrated.

With particular reference to FIGS. 1 and 12-18, the lower open or split friction fit retainer 12, that operates to capture the shank upper portion 8 within the receiver 10 is shown. In certain stages of assembly and operation, the retainer 12 is fully constrained within the receiver, being captured within the receiver cavity 61 at a location below the surface 96 with resilient spring tabs thereof captured in the receiver apertures 78, the retainer 12 not substantially rotatable and not pivotable with respect to the receiver and not readily removable out of the receiver once deployed downward into the receiver cavity 61. The retainer 12 has a central axis that is operationally the same as the axis B associated with the receiver 10 when the shank upper portion 8 and the retainer 12 are installed within the receiver 10. The retainer 12 includes a substantially annular, cylindrical and discontinuous body 115. Extending upwardly and outwardly from the body 115, and integral thereto, are a pair of opposed, resilient, spring tabs 118. The body 115 includes a discontinuous upper or top planar surface 120 and a discontinuous substantially planar bottom surface 121. The spring tabs 118 extend upwardly and outwardly from the top surface 120. Outer surfaces of the retainer body 115 include a discontinuous upper frusto conical surface 123 located adjacent the top surface 120 and a discontinuous lower cylindrical surface 124 adjacent to the frusto-conical surface 123 and extending to or near the bottom surface 121. In some embodiments, a curved or chamfered surface 125 connects the surface 124 with the bottom surface 121. The illustrated embodiment further includes four grooves 127 formed in the surfaces 123 and 124 and running from the top surface 120 through the bottom surface 121. In the illustrated embodiment, two grooves 127 are located on each side of the spring tab 118 that is generally disposed opposite a slit or gap, generally 128, also running from the top surface 120 through the bottom surface 121 of the retainer body 115, the slit 128 being located near the opposing spring tab 118. The grooves 127 provide additional resiliency during loading and manipulation of the retainer 12 with respect to the receiver 10 and the shank head 8. It is foreseen that fewer or greater numbers of grooves 127 may be used. Also, if a greater number of grooves are used, such grooves may be of a reduced size as compared to the illustrated grooves 127. In some embodiments, no grooves may desired. The lower outer cylindrical surface or skirt 124 and the bottom surface 121 are receiver seating surfaces as will be described in greater detail below. In the illustrated embodiment, transition areas where the body 115 meets the spring tabs 118 or the retainer bottom 121 are curved or chamfered.

The opposed resilient spring tabs 118 each have a curved or radiused outer surface 130 (may be substantially cylindrical or have more than one radius), a substantially planar inner surface 131 and a planar top surface 132 that is substantially parallel to the retainer bottom surface 121 when the retainer 12 is in a neutral or nominal state. Near the top surface 132 the inner surface 131 includes a cut-out or step, forming a planar step surface 133 and a curved or cylindrical inner surface 134 located adjacent the top surface 132. The surfaces 133 and 134 provide additional clearance with respect to the insert 14 during shipping and assembly as will be described in greater detail below. The tabs or panels 118 generally extend upwardly and outwardly away from the retainer body 115 when in a neutral or nominal state but may be pressed toward one another and placed in resilient contact with inner surfaces of the receiver 10 during shipping and assembly.

The retainer ring 12 is made from a resilient material, such as a stainless steel or titanium alloy, so that the retainer 12 body 115 may be expanded and the tabs 118 of the retainer may be manipulated during various steps of assembly as will be described in greater detail below. The retainer 12 has a central channel or hollow through bore, generally 141, that passes entirely through the retainer 12 from the spring tab top surfaces 132 to the bottom surface 121 of the retainer body 115. Surfaces that define the channel or bore 141 at the body 115 include a discontinuous inner lower frusto-conical surface 143 adjacent to the retainer body bottom surface 121, a discontinuous, substantially cylindrical surface 145 adjacent the frusto-conical surface 143 and a discontinuous substantially spherical surface 146 located adjacent the cylindrical surface 145. The spherical surface 146 extends upwardly to the retainer body top surface 120. An edge 147 defined by the juncture of the spherical surface 146 and the cylindrical surface 145 provides a component of a locking structure for the assembly 1. The inner concave spherical surface 146 has a radius the same or substantially similar to a radius of the shank head surface 34. An upper portion of the spherical surface 146 located near the body top surface 120 and identified herein as 146′ is ultimately located above the shank head hemisphere 40 and initially frictionally engages the shank head surface 34 to provide a friction fit against the surface 34, allowing the head 8 to be slidingly moveable with respect to the retainer 12, but in a non-floppy manner. However, once a downward pressure is placed on the shank head 8 by the insert 14, the retainer is pressed downwardly and outwardly from the shank, with the shank being primarily locked against the retainer at and near the retainer edge 147, but not at the spherical surface 146′ that is located above the shank hemisphere 40.

The slit 128 creates a split or open ring retainer 12, the slit cutting entirely through the retainer body 115. In the illustrated embodiment, the slit runs at an obtuse angle with respect to the top 120 and bottom 121 retainer body surfaces. In some embodiments, the slit may run substantially perpendicular to the surface 120 and 121. The slit 128 is primarily for expansion of the retainer 12 during pop-on or snap-on assembly with the shank head 8. However, the slit 128 also compresses during assembly with the receiver 10 as will be described in greater detail below. The slit 128 is located near one of the spring tabs 118. Furthermore, at the location of the slit 128, a discontinuous curved concave, cut-out surface 149 and 149′ is formed in the bottom surface 121, the frusto-conical surface 143, the cylindrical surface 145 and up into a portion of the spherical surface 146. The surface 149 and 149′ is radiused or otherwise curved for engagement with the shank head 8 at the surface 34 as will be described in greater detail below. In the illustrated embodiment, the cut-out surface portion 149 on one side of the slit 128 is larger than the surface portion 149′ located on the other side of the slit 128 to align with the receiver cupped portion 109 and thus provide for a desirable increased angle of orientation between the shank 8 and the retainer 12 and thus a desirable increased angle of articulation between the shank 8 and the receiver 10. The through slit 128 of the resilient retainer 12 is also defined by first and second end surfaces, 152 and 153 disposed in substantially parallel spaced relation to one another when the retainer is in a neutral or nominal state. Both end surfaces 152 and 153 are disposed at an obtuse angle with respect to the body top surface 120, but in some embodiments may be disposed perpendicular thereto. A width between the surfaces 152 and 153 is narrow to provide stability to the retainer 12 during operation, but wide enough to allow for some compression of the retainer during assembly as will be described in greater detail below. Because the retainer 12 is top loadable in a substantially neutral state and ultimately expands during locking of the polyaxial mechanism, the width of the slit 128 may be much smaller than might be required for a bottom loaded compressible retainer ring.

With particular reference to FIGS. 1 and 19-26, the locking compression insert 14 is illustrated that is sized and shaped to be received by and down-loaded into the receiver 10 at the upper opening 66. The compression insert 14 has an operational central axis that is the same as the central axis B of the receiver 10. In operation, the insert advantageously frictionally engages the bone screw shank upper portion 8 as well as engaging the receiver 10 in an interference fit engagement, locking the shank 4 in a desired angular position with respect to the receiver 10 that remains in such locked position even if, for example, a rod and closure top are later removed and the rod is replaced with another rod or other longitudinal connecting member or member component, such as a sleeve of a tensioned cord connecting member. Such locked position may also be released by the surgeon if desired with insert engaging tools (not shown). As will be described in greater detail below with respect to the alternative insert 14″ shown in FIGS. 54-55, in some embodiments of the invention, the insert does not have the receiver interference fit feature. The locking insert 14 and the non-locking insert 14″ are preferably made from a solid resilient material, such as a stainless steel or titanium alloy, so that portions of the insert may be grasped, pinched or pressed, if necessary, and un-wedged from the receiver 10 with a release tool (not shown).

The locking compression insert 14 includes a body 156 with cylindrical surfaces of a variety of diameters as well as planar surfaces and chamfers and cut-outs to provide clearance between the insert 14 and the retainer 12 during various steps of assembly as well as when the assembly 1 is in a final locked position. The body 156 is integral with a pair of upstanding arms 157. Located between the arms 157, the body 156 has an outer partial cylindrical surface 158. Each arm outer surface is substantially cylindrical in profile but is made from a variety of facets or faces as well as cut-outs to provide for clearance with other components of the assembly 1. Located on each upstanding arm 157 is a substantially vertical interference fit surface, face or bar 159 that extends outwardly from faceted outer surfaces 160, the bar 159 having an outer slightly curved or cylindrical surface, the bar centrally located on the arm 157 and running substantially parallel to a central axis of the insert 14. Thus a diameter of the insert 14 measured at the surface 159 is larger than a diameter measured at surfaces 160. Beneath each surface 159 and arm surfaces 160 is a discontinuous cylindrical surface 161 having a diameter slightly smaller or the same or similar to the diameter of the arms measured at surfaces 160. A planar lower ledge surface 162 is adjacent to the surface 161 and perpendicular thereto. Adjacent the ledge 162 is a planar inset surface 163 that also is perpendicular to the ledge surface 162, the surface 163 extending downwardly toward a bottom surface 164 of the insert 14. In the illustrated embodiment, the bottom surface 164 is a narrow annular rim that thickens somewhat at front and rear corners of the insert. The insert 14 further includes substantially planar arm top surfaces 165 located opposite the bottom surface 164. The arms 157 are each further defined by substantially planar front and rear surfaces 166 the run from the top surfaces 165 to the bottom surface 164. At the bottom surface 164, the front and rear surfaces 166 are quite narrow due to clearance cut-outs 167 located at each corner of the insert 14.

The arms 157 are sized and configured for ultimate placement at or beneath the cylindrical run-out surface 90 located below the receiver guide and advancement structure 72. Located on the arms 157 and extending outwardly from the arms surfaces 160 and at the top surfaces 165 are a pair of opposed extensions or wings 168. The wings 168 are partially defined by the upper surfaces 165, by outer partially cylindrical surfaces 170, by lower surfaces 171 and opposed planar side surfaces 172, the upper surfaces 169 and the lower surfaces 171 being substantially parallel to on another. In the illustrated embodiment, other chamfered and angled surfaces also define lower portions of the wings 168. The opposed side surfaces 172 generally span between top and bottom surfaces 165 and 171 respectively, of each wing 168, the side surfaces 172 being substantially perpendicular to adjacent top and bottom surfaces 169 and 171. The cylindrical surfaces 170 are sized and shaped for sliding rotation within the receiver arm cylindrical surfaces 90 during assembly of the insert 14 with the receiver 10 as will be described in greater detail below.

Returning to the inner surfaces of the insert 14, a through bore, generally 173, is disposed primarily within and through the body 156 and communicates with a generally U-shaped through channel formed by a saddle surface 174 that is substantially defined by the upstanding arms 157. Near the top surfaces 165, the saddle surface 174 is substantially planar. The saddle 174 has a curved lower seat 175 sized and shaped to closely, snugly engage the rod 21 or other longitudinal connecting member. It is foreseen that an alternative embodiment may be configured to include planar holding surfaces that closely hold a square or rectangular bar as well as hold a cylindrical rod-shaped, cord, or sleeved tensioned cord longitudinal connecting member. A pair of opposed, inwardly facing apertures 176 are located in the saddle 174 beginning near a juncture of the substantially planar upper portion of the saddle 174 and extending into the curved lower seat 175. The grooves 176 are sized and shaped to receive tooling for rotation, locking, unlocking and other manipulation of the insert 14.

The bore, generally 173, is substantially defined at the body 156 by an inner cylindrical surface 177 that communicates with the seat 175 and also communicates with a lower concave, radiused or otherwise curved portion 178 having shank gripping surfaces or ridges 180, the portion 178 generally having a radius for closely mating with the surface 34 of the shank upper portion 8. The portion 178 terminates at the base surface 164. In the illustrated embodiment, the gripping surfaces or ridges 180 are located near the cylindrical surface 177 and a lower part of the portion 178 is a smooth, spherical surface. In other embodiments, the gripping ridges are located all along the surface 178. The gripping ridges or steps 180 are sized and shaped to grip and penetrate into the shank head 8 when the insert 14 is locked against the head surface 34. It is foreseen that there may be more or fewer steps or ridges 180. It is foreseen that the gripping ridges 180 as well as a remainder of the lower shank engaging portion 178 may additionally or alternatively include a roughened or textured surface or surface finish, or may be scored, knurled, or the like, for enhancing frictional engagement with the shank upper portion 8.

The compression insert 14 through bore 173 is sized and shaped to receive a driving tool (not shown) therethrough that engages the shank drive feature 46 when the shank body 6 is driven into bone with the receiver 10 attached. Also, in some locking embodiments of the invention, the bore receives a manipulation tool (not shown) used for releasing the insert from a locked position with the receiver, the tool pressing down on the shank and also gripping the insert at the apertures 176, or with other tool engaging features. Each of the arms 157 and the insert body 156 may include more surface features, such as cut-outs notches, bevels, etc. to provide adequate clearance for inserting the insert 14 into the receiver and cooperating with the retainer 12 during the different assembly steps as will be described in greater detail below.

The insert body 156 cylindrical surface 158 has a diameter slightly smaller than a diameter between crests of the guide and advancement structure 72 of the receiver 10, allowing for top loading of the compression insert 14 into the receiver opening 66, with the arms 157 of the insert 14 being located between the receiver arms 62 during insertion of the insert 14 into the receiver 10. Once the arms 157 of the insert 14 are generally located beneath the guide and advancement structure 72, the insert 14 is rotated into place about the receiver axis B with the wings 168 entering the receiver groove formed by the cylindrical surface 90, the adjacent upper annular surface 91 and the adjacent lower annular surface 92 until the wings are located in the apertures 77 as will be described in greater detail below.

With reference to FIGS. 1, 41, 42 and 46-53, the illustrated elongate rod or longitudinal connecting member 21 (of which only a portion has been shown) can be any of a variety of implants utilized in reconstructive spinal surgery, but is typically a cylindrical, elongate structure having the outer substantially smooth, cylindrical surface 22 of uniform diameter. The rod 21 may be made from a variety of metals, metal alloys, non-metals and deformable and less compressible plastics, including, but not limited to rods made of elastomeric, polyetheretherketone (PEEK) and other types of materials, such as polycarbonate urethanes (PCU) and polyethelenes.

Longitudinal connecting members for use with the assembly 1 may take a variety of shapes, including but not limited to rods or bars of oval, rectangular or other curved or polygonal cross-section. The shape of the insert 14 may be modified so as to closely hold the particular longitudinal connecting member used in the assembly 1. Some embodiments of the assembly 1 may also be used with a tensioned cord. Such a cord may be made from a variety of materials, including polyester or other plastic fibers, strands or threads, such as polyethylene-terephthalate. Furthermore, the longitudinal connector may be a component of a longer overall dynamic stabilization connecting member, with cylindrical or bar-shaped portions sized and shaped for being received by the compression insert 14 of the receiver having a U-shaped, rectangular- or other-shaped channel, for closely receiving the longitudinal connecting member. The longitudinal connecting member may be integral or otherwise fixed to a bendable or damping component that is sized and shaped to be located between adjacent pairs of bone screw assemblies 1, for example. A damping component or bumper may be attached to the longitudinal connecting member at one or both sides of the bone screw assembly 1. A rod or bar (or rod or bar component) of a longitudinal connecting member may be made of a variety of materials ranging from deformable plastics to hard metals, depending upon the desired application. Thus, bars and rods may be made of materials including, but not limited to metal and metal alloys including but not limited to stainless steel, titanium, titanium alloys and cobalt chrome; or other suitable materials, including plastic polymers such as polyetheretherketone (PEEK), ultra-high-molecular weight-polyethylene (UHMWP), polyurethanes and composites, including composites containing carbon fiber, natural or synthetic elastomers such as polyisoprene (natural rubber), and synthetic polymers, copolymers, and thermoplastic elastomers, for example, polyurethane elastomers such as polycarbonate-urethane elastomers.

With reference to FIGS. 1, 41, 42 and 46-53, the closure structure or closure top 18 shown with the assembly 1 is rotatably received between the spaced arms 62 of the receiver 10. It is noted that the closure 18 top could be a twist-in or slide-in closure structure. The illustrated closure structure 18 is substantially cylindrical and includes a an outer helically wound guide and advancement structure 182 in the form of a flange that operably joins with the guide and advancement structure 72 disposed on the arms 62 of the receiver 10. The flange form may take a variety of forms, including those described in Applicant's U.S. Pat. No. 6,726,689, which is incorporated herein by reference. Although it is foreseen that the closure structure guide and advancement structure could alternatively be a buttress thread, a square thread, a reverse angle thread or other thread like or non-thread like helically wound advancement structure, for operably guiding under rotation and advancing the closure structure 18 downward between the arms 62 and having such a nature as to resist splaying of the arms 62 when the closure structure 18 is advanced into the channel 64, the flange form illustrated herein as described more fully in Applicant's U.S. Pat. No. 6,726,689 is preferred as the added strength provided by such flange form beneficially cooperates with and counters any reduction in strength caused by the any reduced profile of the receiver 10 that may more advantageously engage longitudinal connecting member components. The illustrated closure structure 18 also includes a top surface 184 with an internal drive 186 in the form of an aperture that is illustrated as a star-shaped internal drive such as that sold under the trademark TORX, or may be, for example, a hex-shaped drive or other internal drives such as slotted, tri-wing, spanner, two or more apertures of various shapes, and the like. A driving tool (not shown) sized and shaped for engagement with the internal drive 186 is used for both rotatable engagement and, if needed, disengagement of the closure 18 from the receiver arms 62. It is also foreseen that the closure structure 18 may alternatively include a break-off head designed to allow such a head to break from a base of the closure at a preselected torque, for example, 70 to 140 inch pounds. Such a closure structure would also include a base having an internal drive to be used for closure removal. A base or bottom surface 188 of the closure is planar and further includes a point 189 and a rim 190 for engagement and penetration into the surface 22 of the rod 21 in certain embodiments of the invention. It is noted that other embodiments may or may not include the point and/or the rim. The closure top 18 may further include a cannulation through bore (not shown) extending along a central axis thereof and through the top and bottom surfaces thereof. Such a through bore provides a passage through the closure 18 interior for a length of wire (not shown) inserted therein to provide a guide for insertion of the closure top into the receiver arms 62.

An alternative closure top, such as the top 18′ shown in FIGS. 43 and 44 for use with a deformable rod, such as a PEEK rod 21′, for example, includes a bottom surface 188′ that has domed portion 190′ with a central nub 189′ in lieu of the point and rim surface of the closure top 18. Otherwise, the closure top 18′ includes a guide and advancement structure 182′, a top surface 184′ and an internal drive feature 186′ the same or substantially similar to the respective guide and advancement structure 182, top surface 184 and internal drive feature 186 of the closure top 18.

The assembly 1 receiver 10, retainer 12 and compression insert 14 are typically assembled at a factory setting that includes tooling for holding and alignment of the component pieces and manipulating the retainer 12 and the insert 14 with respect to the receiver 10. In some circumstances, the shank 4 is also assembled with the receiver 10, the retainer 12 and the compression insert 14 at the factory. In other instances, it is desirable to first implant the shank 4, followed by addition of the pre-assembled receiver, retainer and compression insert at the insertion point. In this way, the surgeon may advantageously and more easily implant and manipulate the shanks 4, distract or compress the vertebrae with the shanks and work around the shank upper portions or heads without the cooperating receivers being in the way. In other instances, it is desirable for the surgical staff to pre-assemble a shank of a desired size and/or variety (e.g., surface treatment of roughening the upper portion 8 and/or hydroxyapatite on the shank 6), with the receiver, retainer and compression insert. Allowing the surgeon to choose the appropriately sized or treated shank 4 advantageously reduces inventory requirements, thus reducing overall cost and improving logistics and distribution.

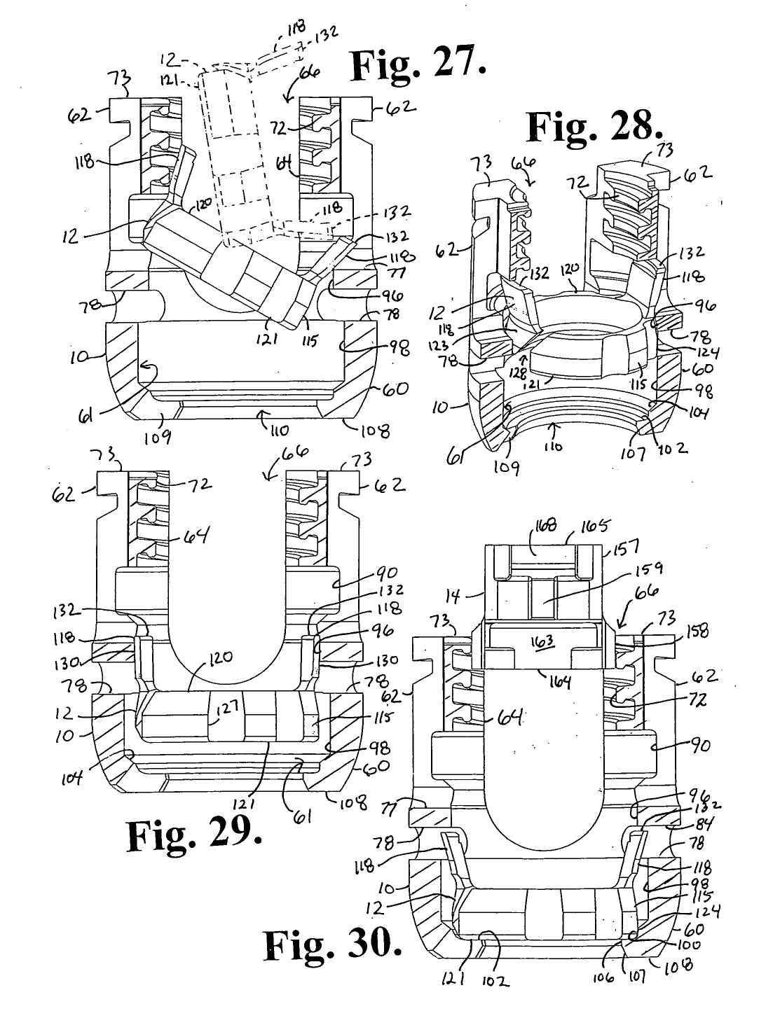

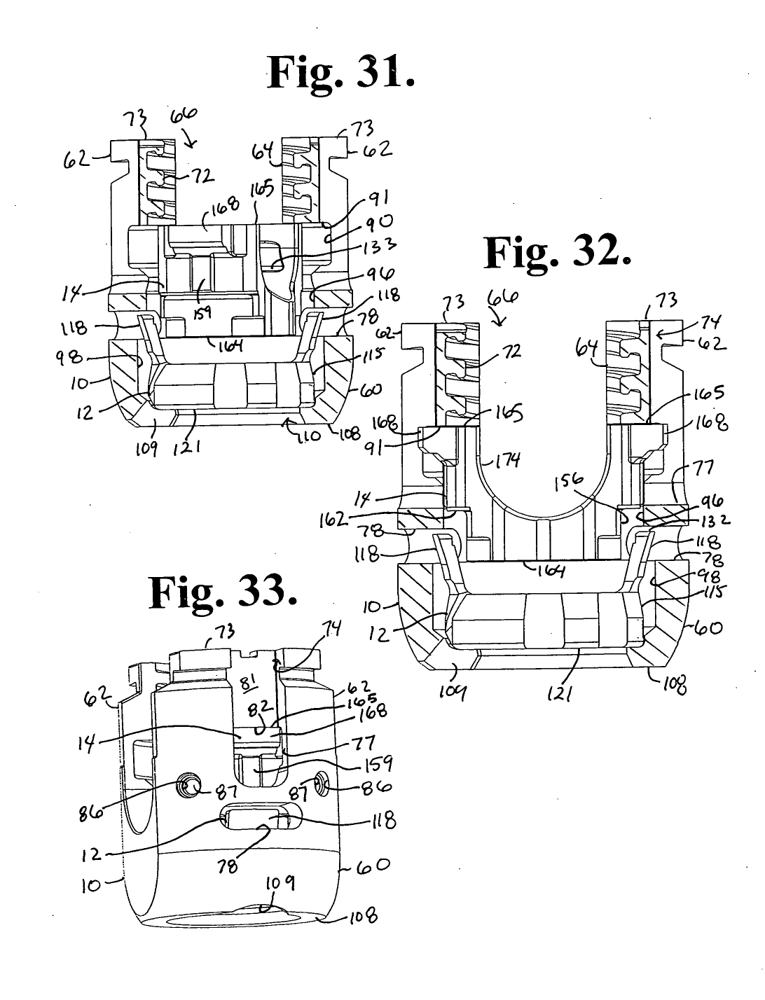

Pre-assembly of the receiver 10, retainer 12 and compression insert 14 is shown in FIGS. 27-35. With particular reference to FIG. 27, first the retainer 12 is inserted into the upper receiver opening 66, leading with the spring tab 118 with the tab 118 top surface 132 facing one arm 62 and the retainer bottom surface 121 facing the opposing arm 62 (shown in phantom). The retainer 12 is then lowered in such sideways manner into the channel 64 and partially into the receiver cavity 61, followed by tilting the retainer 12 such that at least one of the spring tabs 118 is received into one of the apertures 77 as the opposed tab 118 is positioned beneath the guide and advancement structure 72. Then, with reference to FIG. 28, the retainer 12 is tilted into a position wherein the central axis of the retainer 12 is generally aligned with the receiver central axis B. As shown in FIG. 28, the retainer outer surfaces 124 engage the receiver inner cylindrical surface 96 and the retainer slit 128 is reduced such that the surfaces 152 and 153 that define the slit 128 are pressed toward one another to a touching or almost touching state while the surfaces 124 are slid past the receiver surface 96. With reference to FIG. 29, the retainer 12 is pressed downwardly into the receiver to a location wherein the spring tabs 118 resiliently press against the receiver surface 96, holding the retainer within the receiver cavity 61 at a temporary position, if desired. At this time, the retainer 12 is not yet fully captured within the receiver base cavity 61, but cannot be readily removed unless the tabs 118 are squeezed toward one another using a tool or tools.

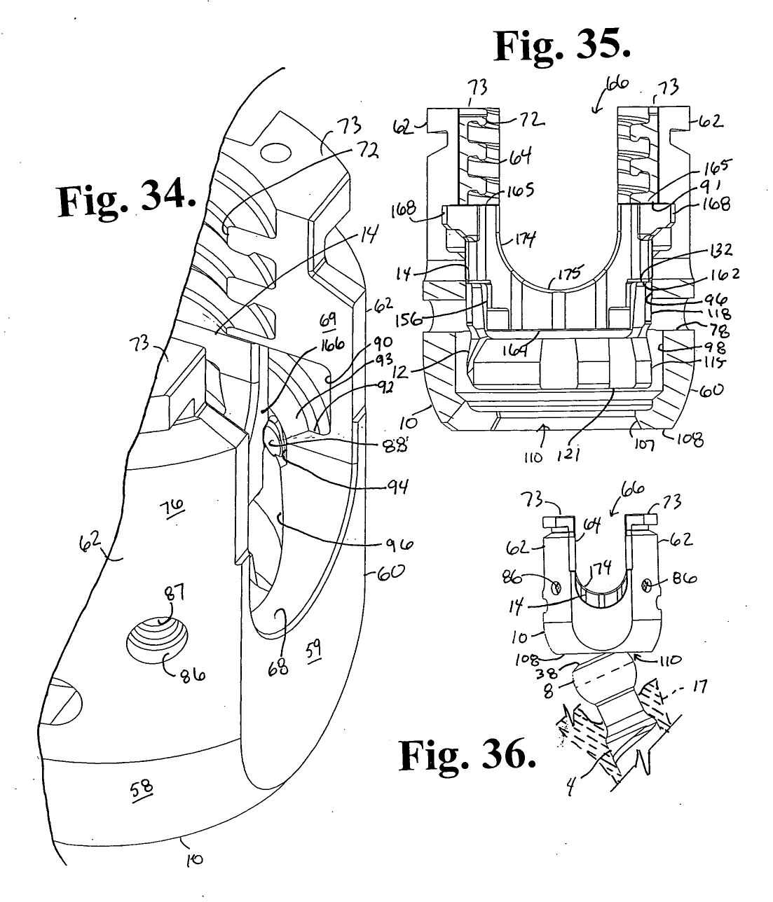

With further reference to FIG. 30, the retainer 12 may then be pressed further downwardly into a seated position within the receiver with the spring tabs 18 expanding to a relaxed or nominal state into the apertures 78 and the retainer bottom surface 121 seated on the receiver annular surface 102. Also with reference to FIG. 30, at this time, the compression insert 14 is then downloaded into the receiver 10 through the upper opening 66 with the bottom surface 164 facing the receiver arm top surfaces 73 and the insert arm wings 168 located between the opposed receiver arms 62. The insert 14 is then lowered toward the receiver base 60 until the insert 14 arm upper surfaces 165 are adjacent the run-out area below the guide and advancement structure 72 defined in part by the cylindrical surface 90. With reference to FIG. 31, thereafter, the insert 14 is rotated about the receiver axis B until the upper arm surfaces 165 are directly below the guide and advancement structure 72 with the U-shaped channel 174 of the insert 14 aligned with the U-shaped channel 64 of the receiver 10 and the insert wings 168 located at the apertures 77 as shown in FIG. 32. In some embodiments, the insert arms may need to be compressed slightly during rotation to clear some of the inner surfaces 70 of the receiver arms 62. With particular reference to FIGS. 33 and 34, at this time, the four crimping wall portions 87 are pressed inwardly towards the insert 14 and crimping wall material 88 thus engages the insert at front and rear surfaces 166 as best shown in FIG. 34. The crimping wall material 88 of the wall 87 pressing against the insert 14 at a total of four locations thereby prohibits the insert 14 from rotating with respect to the receiver axis B. At this time, there can be some upward and downward movement of the insert 14, but such movement is limited as the upper wall 82 defining the receiver aperture 77 stops further upward movement of the insert wings 168. Also, as shown in FIG. 35, the retainer tabs 118 are then moved inwardly and the retainer is moved upwardly until the retainer tab top surfaces 132 abut against insert surfaces 162. The resilient retainer tabs 118 also press outwardly against the receiver surfaces 96 and thus the retainer 12 stops any downward movement of the now trapped insert 14. The retainer 12 and the insert 14 are now in a desired position for shipping as an assembly along with the separate shank 4.

Typically, the receiver and retainer combination are shipped or otherwise provided to the end user with the spring tabs 118 wedged against the receiver as shown in FIG. 35. The receiver 10, retainer 12 and insert 14 combination is now pre-assembled and ready for assembly with the shank 4 either at the factory, by surgery staff prior to implantation, or directly upon an implanted shank 4 as will be described herein.

As illustrated in FIG. 36, the bone screw shank 4 or an entire assembly 1 made up of the assembled shank 4, receiver 10, retainer 12 and compression insert 14, is screwed into a bone, such as the vertebra 17 (shown in phantom), by rotation of the shank 4 using a suitable driving tool (not shown) that operably drives and rotates the shank body 6 by engagement thereof at the internal drive 46. Specifically, the vertebra 17 may be pre-drilled to minimize stressing the bone and have a guide wire (not shown) inserted therein to provide a guide for the placement and angle of the shank 4 with respect to the vertebra. A further tap hole may be made using a tap with the guide wire as a guide. Then, the bone screw shank 4 or the entire assembly 1 is threaded onto the guide wire utilizing the cannulation bore 50 by first threading the wire into the opening at the bottom 28 and then out of the top opening at the drive feature 46. The shank 4 is then driven into the vertebra using the wire as a placement guide. It is foreseen that the shank and other bone screw assembly parts, the rod 21 (also having a central lumen in some embodiments) and the closure top 18 (also with a central bore) can be inserted in a percutaneous or minimally invasive surgical manner, utilizing guide wires and attachable tower tools mating with the receiver. When the shank 4 is driven into the vertebra 17 without the remainder of the assembly 1, the shank 4 may either be driven to a desired final location or may be driven to a location slightly above or proud to provide for ease in assembly with the pre-assembled receiver, compression insert and retainer.

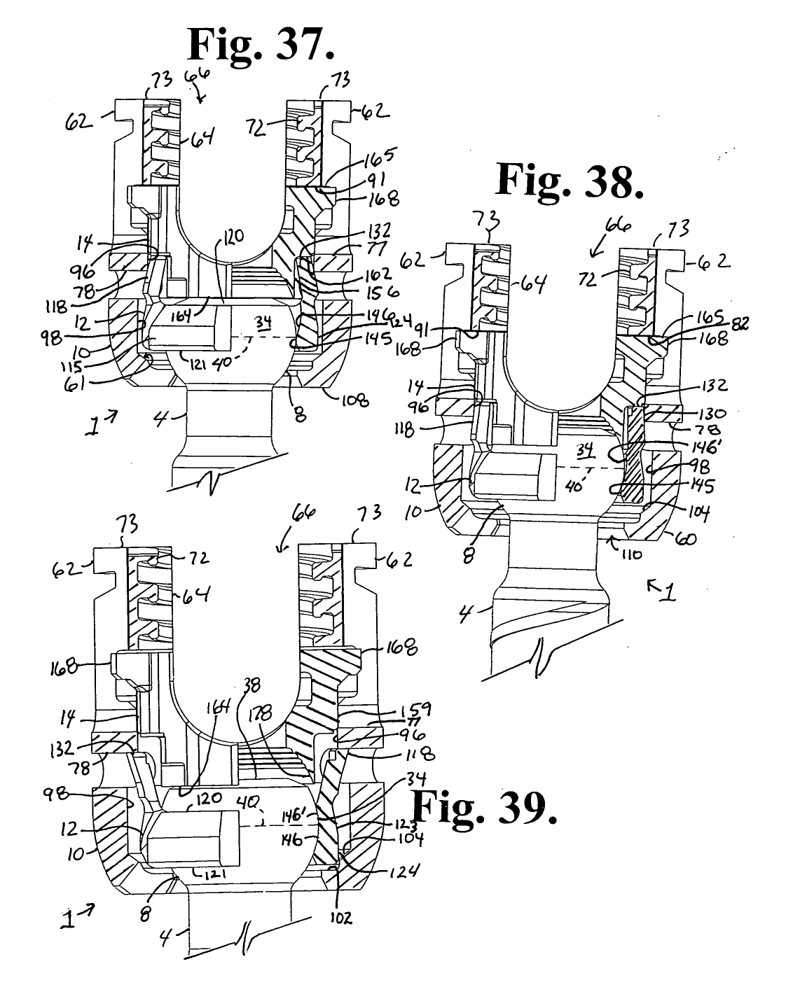

With reference to FIGS. 36 and 37, the pre-assembled receiver, insert and retainer are placed above the shank upper portion 8 until the shank upper portion is received within the opening 110. With particular reference to FIGS. 37-39, as the shank upper portion 8 is moved into the interior 61 of the receiver base, the shank upper portion 8 presses upwardly against the retainer 12 in the receiver recess partially defined by the cylindrical surface 98, specifically the surface portions 124 are pressed outwardly toward the surface 98 as the retainer 12 expands about the shank 8. As the shank head 8 continues to move upwardly toward the channel 64, the shank head surface 34 also forces the retainer 12 against the insert 14. However, the insert 14 is prohibited from moving upward by the wings 168 abutting against the surfaces 82 (that is also the ceiling annular surface 91 adjacent the groove 90) defining the apertures 77. Therefore, the upwardly moving shank head 8 forces a widening of the retainer slit 128 and corresponding outward movement of the body 115 of the retainer 12 towards the receiver cylindrical surfaces 98 and stepped surface 104 defining the receiver expansion recess or chamber as best shown in FIG. 37, while the retainer tabs 118 near the top surfaces 134 thereof are generally maintained in a location below the insert 14 arm surfaces 162, with the tabs 118 being pressed inwardly toward the axis B at the termination of the receiver wall surface 96. At this time, the spherical surface 34 of the head 8 comes into contact with the retainer inner cylindrical body 145 and the edge 147. With reference to FIG. 38, the retainer 12 begins to return towards a neutral or nominal state as the center of the sphere 40 of the shank head 8 passes beyond the retainer inner edge 147. By the time the hemisphere of the spherical surface 34 extends into a desired captured location within the retainer central bore 141, the shank surface 34 is in contact with the radiused surface 146 near the edge 147 but not yet in full contact with the upper portion 146′ of the radiused surface. With reference to FIG. 39, the receiver is manually pulled upwardly or the shank 4 and attached retainer 12 are moved manually downwardly as far as possible. Now, the resilient spring tabs 118 snap out into the receiver apertures 78 and the resilient retainer 12 fully contacts the shank head 8 at the surface 34 with the upper radiused portion 146′ snapping onto the shank head above the hemisphere 40, resiliently pressing against the radiused surface 34 and providing a fairly tight friction fit between the head 8 and the retainer 12, the surface 34 being pivotable with respect to the retainer 12 with some force. Thus, a tight, non-floppy ball and socket joint is now created between the retainer 12 and the shank upper portion 8 even before the retainer 12 is fully seated within the receiver as shown in FIG. 39. The now neutral spring tabs 118 are also fully captured within the receiver apertures 78 and the retainer, as well as the captured shank, is now fully limited by the apertures with respect to upward and downward movement as well as rotation with respect to the receiver.

As noted above, FIG. 39 shows the assembly after the receiver is manually pulled upwardly or the shank 4 and attached retainer 12 are moved manually downwardly as far as possible. Full seating of the retainer on the receiver surface 102 does not typically occur unless the assembly is forced downwardly by tooling (not shown) as illustrated in FIG. 40, or by the closure top 18 pressing the rod 21 downwardly against the insert 14 as shown in FIGS. 41 and 42. The retainer lower bottom surface 121 is seated on the receiver annular surface 102. Downward pressure of the shank head 8 on the retainer edge 147 further expands the retainer body 115 outwardly, with the retainer outer surface 124 pressing against the receiver inner cylindrical surface 100. The retainer body formed in part by the lower skirt surface 124 advantageously allows for the head 8 to seat lower within the receiver than in other known polyaxial bone anchors.

With further reference to FIGS. 40-42, after the retainer 12 is moved downwardly into the receiver 10 into the position shown in FIG. 39, the insert 14 remains located spaced above the shank head 8 as the outer surfaces 159 rest upon the receiver cylindrical surfaces 96, prohibiting downward movement of the insert 14 unless a downward force is applied on the insert either by a tool or tools at the wings 168 and/or the inner apertures 176 or the insert 14 s pressed downwardly by the rod 21 and closure top 18.

In some instances, a tool or tools are used to press the insert downwardly and the surfaces 159 into locking engagement with the receiver surfaces 96 as shown in FIG. 40, prior to implanting the entire assembly. It may then be desirable to use a tool or tools to pull the insert 14 away from locking engagement with the shank head surface 34 to allow for polyaxial manipulation of the shank 4 with respect to the receiver 10. After the insert 14 is pulled upwardly, the retainer remains fully seated, but with the insert now disengaged from the shank head 8, a friction fit returns between the shank surface 34 above the hemisphere 40 and the upper portion 146′ of the radiused surface of the retainer 12, as shown, for example, in FIG. 45. In other instances, tools may be used to pull the insert 14 up only slightly, so that the insert 14 now provides only a friction fit against the shank head 8 and therefore the shank may be pivoted to a desired position with some force. Tools that may be used for such an arrangement are described for example, in Applicants' U.S. Provisional Patent App. 61/626,250 filed Sep. 23, 2011 and incorporated by reference herein.

In some embodiments, when the receiver 10 is pre-assembled with the shank 4, the entire assembly 1 may be implanted by inserting the driving tool (not shown) into the receiver and the shank drive 46 and rotating and driving the shank 4 into a desired location of the vertebra 17. At such time, prior to locking with a closure top, the receiver 10 may be articulated to a desired angular position with respect to the shank 4(such as the angular orientations shown in FIGS. 46-53, for example), that will be held, but not locked, by the frictional engagement between the retainer 12 inner radiused surface 146′ and the shank upper portion 8. In some cases it may be desirable to lock the insert 14 into the receiver 10 at this time, the insert 14 being pressed downwardly into locking engagement with the shank head 8 by a tool pressing downwardly on the insert, for example, with a tool (not shown) entering through the receiver outer grooves 74 and pressing downwardly on the insert wings 168. Such a tool may also include (or alternatively be) a structure for gripping the receiver, for example, a pronged tool or tool portion with some of the tool extending into the receiver channel 64. Or, as explained above, the insert 14 may remain spaced above the shank head 8 until locked into place by the rod 21 and the closure top 18 pressing down upon the insert 14.

As explained above, the diameter of the insert at the outer surface or band 159 is sized large enough to require that the surface 159 must be forced into the cylindrical surface 96 of the receiver by a tool or tools or by the closure top 18 forcing the rod 21 downwardly against the insert 14 with sufficient force to interferingly frictionally lock or wedge the insert 14 into the receiver 10 at the surface 159. This independent lock-and-release feature gives the surgeon flexibility to loosen the closure top and even remove the closure top and rod without affecting the locking of the polyaxial mechanism of the assembly 1, the anchor assembly functioning like a fixed monoaxial screw with the shank 4 in fixed relation with the receiver 10, but with the shank remaining in a desired angle with respect to the receiver. Thus, once a locking insert is in an interference fit locking engagement with the receiver as shown in FIGS. 42 and 43, if a rod and closure top have been assembled with the receiver 10, the closure top 18 may be loosened or removed and/or the rod 21 may be adjusted and/or removed and the frictional engagement between the insert 14 and the receiver 10 at the receiver surface 96 will remain locked in place, advantageously maintaining a locked angular position of the shank 4 with respect to the receiver 10. At such time, another rod, such as a deformable rod 21′ and cooperating alternative closure top 18′ shown in FIG. 43 may be loaded onto the already locked-up assembly to result in an alternative assembly shown in FIG. 44.

If unlocking of the insert 14 with respect to the receiver 10 is desired, a tool (not shown) may be inserted into the through apertures 77 below the insert wings 168 and the insert 14 may be pulled away from the receiver 10. Such a tool may include a piston-like portion for pushing directly on the shank while the insert 14 is pulled away from the receiver. At such time, the shank 4 may be articulated with respect to the receiver 10, and the desired friction fit returns between the retainer 12 and the shank surface 34, so that an adjustable, but non-floppy relationship still exists between the shank 4 and the receiver 10. If further disassembly if the assembly is desired, such is accomplished in reverse order to the procedure described previously herein for the assembly 1.

Returning to FIGS. 40-42, what is illustrated is the locking of the polyaxial mechanism by using tools (not shown) to pressed the insert 14 down onto the shank head 8. However, the assembly as shown in FIG. 39 may be implanted and then locked by positioning the rod 21 in an open or percutaneous manner in cooperation with the at least two bone screw assemblies 1. The closure structure 18 is then advanced between the arms 62 of each of the receivers 10. The closure structure 18 is rotated, using a tool engaged with the inner drive 186 until a selected pressure is reached at which point the rod 21 engages the U-shaped saddle 174 of the compression insert 14, further pressing the insert spherical surface 178 and stepped shank gripping surfaces 180 against the shank spherical surface 34, the edges 180 penetrating into the spherical surface 34, pressing the shank upper portion 8 into locked frictional engagement with the retainer 12. Specifically, as the closure structure 18 rotates and moves downwardly into the respective receiver 10, the rim 190 engage and penetrate the rod surface 22, the closure structure 18 pressing downwardly against and biasing the rod 21 into compressive engagement with the insert 14 that urges the shank upper portion 8 toward the retainer 12 and into locking engagement therewith at the retainer edge surface 147, the retainer 12 frictionally abutting the receiver surface 102 and pressing outwardly against the receiver cylindrical surface 100. For example, about 80 to about 120 inch pounds of torque on the closure top may be applied for fixing the bone screw shank 6 with respect to the receiver 10. At this time, the retainer inner edge 147 engages and digs into the shank head 8. At this time, the retainer inner radius surface 146′ may be slightly spaced from the shank head 8 or may be still touching the shank spherical surface 34, but is no longer in tight or close frictional engagement with the surface 34 and thus are not participating in the final locking engagement between the shank 4 and the retainer 12. If disassembly if the assembly 1 is desired, such is accomplished in reverse order to the procedure described previously herein for assembly.

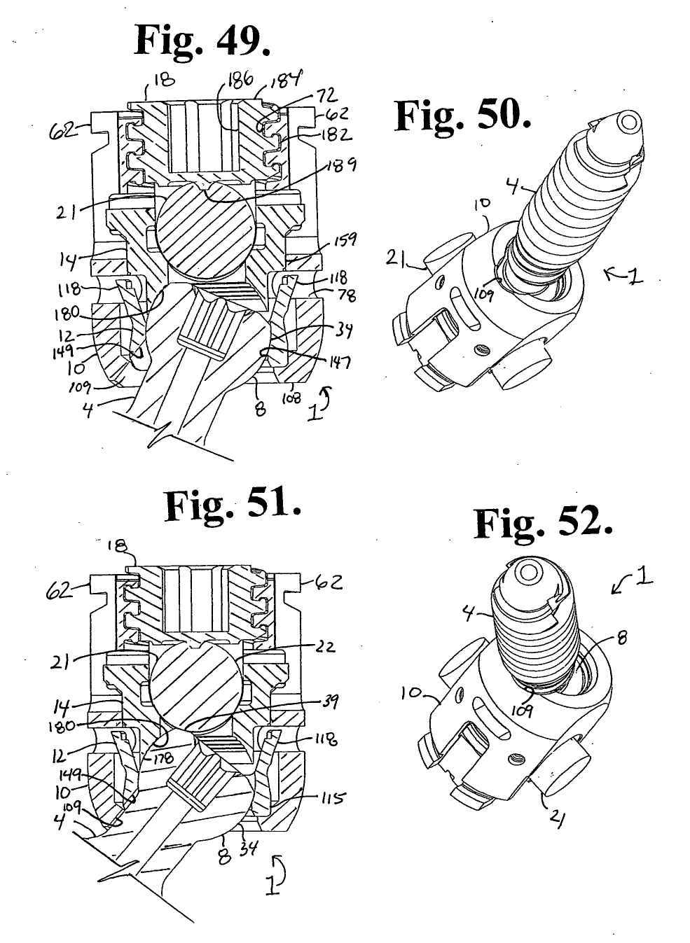

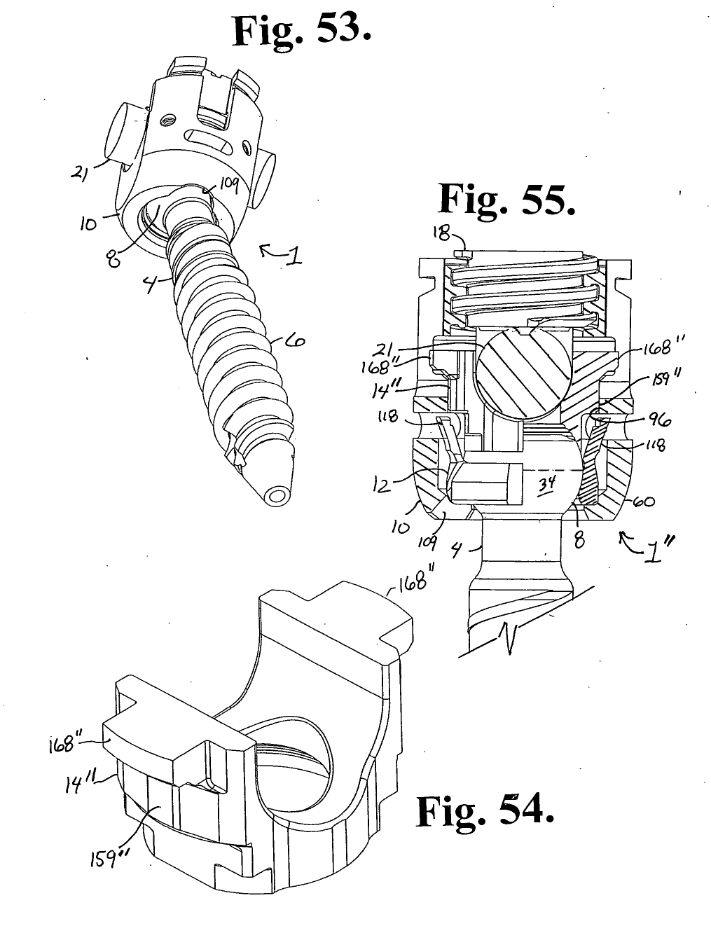

With reference to FIGS. 46-53, different angular or articulated positions of the shank 4 with respect to the receiver 10 are shown, some making full use of the cut-out or cupped surfaces 149 of the retainer 12 and the cupped surface 109 of the receiver 10. For example in FIGS. 51-52 the shank 8 is shown pivoted toward and into engagement with the cupped surfaces 109 and 149 as compared to the arrangement shown in FIGS. 46-48, wherein the shank 4 is pivoted in a direction away from the retainer surface 109. Specifically, in FIGS. 46-48 a twenty-five degree caudad relationship between the shank 4 and the receiver 10 is illustrated. In FIGS. 49-50 a twenty-five degree medial relationship between the shank 4 and the receiver 10 is illustrated. In FIGS. 51-52 a forty degree medial relationship between the shank 4 and the receiver 10 is illustrated. And, in FIG. 53, a twenty-five degree multi-planar relationship between the shank 4 and the receiver 10 is illustrated.

With reference to FIGS. 54-55, an alternative non locking compression insert 14″ is illustrated for use with the shank 4, receiver 10, retainer 12, closure top 18 and rod 21 previously described herein. The insert 14″ is substantially similar to the insert 14 previously described herein, having all the features of the insert 14 with the exception of the through apertures 167 and the enlarged interference fit surface 159. Instead, the insert includes a bar or band 159″ sized to allow the insert 14″ to easily slidingly fit within the receiver surface 96 rather than interferingly fit with such surface. The insert 14″ is assembled with the receiver 10, retainer 12, shank 4, rod 21 and closure top 18 in a manner the same as previously described above with respect to the assembly 1, with the exception that the insert 14″ need not be forced downwardly into a locking interference fit with the receiver 10 when the shank 4 is locked in place. If the closure top 18 is loosened or if the closure top 18 and the rod 21 are removed from the assembly 1, the insert 14″ will also shift upwardly in the receiver 10 and the shank 4 will not remain locked with respect to the retainer 12 and the receiver 10. Tooling (not shown) cooperating with the receiver grooves 74 to press downwardly on wings 168″ of the insert 14″ advantageously provides for a temporary locking of the polyaxial mechanism during surgery, if desired by the surgeon.

It is to be understood that while certain forms of the present invention have been illustrated and described herein, it is not to be limited to the specific forms or arrangement of parts described and shown.

What is claimed and desired to be secured by Letters Patent is as follows:

1. In a bone anchor, the improvement comprising:
a) a shank having a body for fixation to a bone and an integral upper portion having a first curved surface;
b) a receiver having a base and a pair of opposed upstanding arms, the arms defining an open channel, the base defining a chamber and a lower opening, the chamber communicating with both the channel and the lower opening, each arm having a non-centrally located crimping aperture, each crimping aperture partially defined by a crimp wall;
c) an insert disposed within the receiver with each crimp wall being pressed into frictional engagement with the insert for substantially prohibiting rotation of the insert with respect to the receiver, each crimp wall located near one of a front and rear surface of the insert, the insert further having a second curved surface sized and shaped for frictional mating engagement with the shank upper portion first curved surface; and
d) a resilient open retainer having a base, the retainer captured within the chamber and expandable about at least a portion of the shank upper portion and wherein expansion-only locking engagement occurs between the shank upper portion and the retainer base and between the retainer base and the receiver.

2. The improvement of claim 1 wherein each crimping aperture is a first crimping aperture and each receiver arm further includes a second crimping aperture spaced from the respective first crimping aperture, each second crimping aperture partially defined by a crimp wall for a total of four crimp walls being pressed against the insert to substantially prohibit rotation of the insert with respect to the receiver.

3. The improvement of claim 1 wherein the insert has an outer face releasably frictionally locked against the receiver.

4. The improvement of claim 1 wherein the receiver channel is a first channel and the insert has a second channel, the insert being top loaded into the receiver and then rotated into a position above the retainer with the second channel aligned with the first channel.

5. The improvement of claim 1 wherein the retainer base has at least a first planar surface seated on a second planar surface partially defining the receiver chamber.

6. The improvement of claim 1 wherein the retainer base has an outer non-tapered surface in engagement with a second surface partially defining the receiver chamber.

7. The improvement of claim 1 wherein the retainer has a third inner curved surface in a friction fit with the shank upper portion first curved surface during temporary manipulation of the shank with respect to the receiver prior to the final expansion-only locking engagement, the shank upper portion being movable with respect to the retainer with some resistance when a force is applied to the shank to pivot the shank with respect to the receiver.

8. In a bone anchor, the improvement comprising:
a) a shank having a body for fixation to a bone and an integral upper portion having a first curved surface;
b) a receiver having a base and a pair of opposed upstanding arms, the arms defining an open channel, the base defining a chamber and a lower opening, the chamber communicating with both the channel and the lower opening, each arm having a pair of spaced crimping apertures, each crimping aperture located near a perimeter of the respective arm, each crimping aperture partially defined by a crimp wall;
c) an insert disposed within the receiver with each crimp wall being pressed into frictional engagement with the insert substantially prohibiting rotation of the insert with respect to the receiver, the insert having a second curved surface sized and shaped for frictional mating engagement with the shank head; and
d) a resilient open retainer having a base, the retainer captured within the chamber and expandable about at least a portion of the shank upper portion, the retainer having a third curved surface in a friction fit with the shank upper portion during temporary manipulation of the shank with respect to the receiver, the shank upper portion being movable with respect to the retainer with some resistance when a force is applied to the shank to pivot the shank with respect to the receiver, and wherein the retainer is in an expanded state when final locking engagement occurs between the shank upper portion and the retainer base and between the retainer base and the receiver.

9. The improvement of claim 8 wherein the retainer further comprises a pair of opposed outwardly extending resilient tabs and the retainer base has at least a first planar surface and each receiver arm has a lower centrally located through aperture and the receiver base has a second planar surface partially defining the receiver chamber and wherein after the shank head is received through the retainer base, the retainer base first planar surface is seated on the receiver second surface and the resilient tabs expand to a neutral state and are captured within the through apertures.

10. The improvement of claim 8 wherein the insert has an outer projected portion releasably frictionally locked against the receiver.

11. In a bone anchor, the improvement comprising:
a) a shank having a body for fixation to a bone and an integral upper portion having a substantially spherical surface;
b) a receiver having a base and a pair of opposed upstanding arms, the arms defining an open channel, each arm having an outer surface, the base defining a chamber and a lower opening, the chamber communicating with both the channel and the lower opening, each arm having a substantially centrally located through aperture and a pair of spaced lateral crimping apertures formed in the arm outer surface and located on either side of the through aperture, each crimping aperture defined in part by a crimp wall;
c) an insert disposed within the receiver with each crimp wall being pressed into frictional engagement with the insert allowing the insert to be moved vertically with some force but substantially prohibiting rotation of the insert with respect to the receiver, the insert having a curved surface portion sized and shaped for frictional mating engagement with the shank spherical surface, the insert further having a pair of outwardly extending arm portions, each arm portion partially extending through one of the centrally located receiver through apertures, the arm portions being engageable by manipulation tools located at the receiver arm outer surface for movement of the insert both downwardly into contact with the shank head and upwardly away from the shank head; and
d) a resilient open retainer having a base, the retainer captured within the chamber and expandable about at least a portion of the shank upper portion, the retainer having a second curved surface in a friction fit with the shank upper portion spherical surface during temporary manipulation of the shank with respect to the receiver, the shank upper portion being movable with respect to the retainer with some resistance when a force is applied to the shank to pivot the shank with respect to the receiver, and wherein the retainer is in an expanded state when final locking engagement occurs between the shank upper portion and the retainer base and between the retainer base and the receiver.

12. The improvement of claim 11 wherein a shallow groove is formed in each receiver arm outer surface, each groove communicating with one of the through apertures, each groove extending from the respective aperture to a top surface of the receiver arm, each groove having a width for receiving a manipulation tool.

13. The improvement of claim 11 wherein the insert has an outer face releasably frictionally locked against the receiver.

14. The improvement of claim 11 wherein the insert is top loaded into the receiver and then rotated into a position above the retainer.

15. The improvement of claim 11 wherein the retainer base has at least a first planar surface seated on a second planar surface partially defining the receiver chamber.

16. The improvement of claim 11 wherein the retainer base has a first outer non-tapered surface in engagement with a second surface partially defining the receiver chamber.

17. The improvement of claim 11 wherein the retainer further comprises a pair of opposed outwardly extending resilient tabs and the retainer base having at least a first planar surface and the receiver having a second planar surface partially defining the receiver chamber and a second pair of opposed apertures and wherein after the shank head is received through the retainer base, the retainer base first planar surface is seated on the receiver second surface and the resilient tabs expand to a neutral state and are captured within the second pair of receiver apertures.
